(19) United States
(12) Patent Application Publication
(10) Pub. No.: US 2016/0250631 A1
(43) Pub. Date: Sep. 1, 2016

(54) REINFORCED MICROPLATE

(71) Applicant: CORNING INCORPORATED, CORNING, NY (US)

(72) Inventors: ManMan Kang, Fremont, CA (US); Wai Kin Poon, Union City, CA (US)

(21) Appl. No.: 15/150,845

(22) Filed: May 10, 2016

Related U.S. Application Data
(63) Continuation-in-part of application No. PCT/US2015/064585, filed on Dec. 9, 2015.
(60) Provisional application No. 62/090,066, filed on Dec. 10, 2014.

(51) Int. Cl. B01L 3/00; C12Q 1/68

(52) U.S. Cl. CPC B01L 3/50851; C12Q 1/686; B01L 2300/0829; B01L 2300/0848; B01L 2300/04; B01L 2300/0887

(57) ABSTRACT

A reinforced microplate has reinforcing members that enhance stiffness and minimize deformation of the microplate, especially thermally-induced deformation. The reinforcing members include dots, ribs or struts that are integrally formed on a bottom surface of the microplate. In cooperation with the reinforcing members, the microplate frame can include one or more slots that act to disrupt the effects of thermal expansion and limit thermally-induced strain. To provide enhanced sealing utility and reduce sample evaporation, each well may include a raised rim with angled transition surfaces leading to a top surface, preferably in combination with at least one elevated sealing feature extending from an upper deck surface, and being arranged between corners of the deck and the plurality of wells.

This application is a continuation-in-part of and claims the benefit of priority under 35 U.S.C. §111(a) of International Patent Application No. PCT/US2015/064585 filed on Dec. 9, 2015, which claims the benefit of priority under 35 U.S.C. §119 of U.S. Provisional Application Ser. No. 62/090,066 filed on Dec. 10, 2014. The entire contents of each of the foregoing applications is relied upon and incorporated by reference herein in its entirety.

### BACKGROUND

1. Field

The present disclosure relates generally to microtiter plates, also known as microplates, and more particularly to reinforced microplates and their methods of manufacture. The reinforced microplates are adapted for use with automated equipment and can withstand thermal cycling without unacceptable deformation.

2. Technical Background

Polymerase chain reaction (PCR) processes involve the replication of genetic material such as DNA and RNA. In both industry and academia, PCR processes are carried out on a large scale using multi-well microplates (e.g., 8 well strips or 96, 384 or even 1536 well arrays). It is desirable to have an apparatus that allows the PCR process to be performed in an efficient and convenient fashion.

Because of their ease of handling and relatively low cost, microplates are often used for sample containment during the PCR process. Microplates may also be used in other research and clinical diagnostic procedures. Reference is made to FIGS. 1A-1C where there are illustrated different views of an example microplate 100. Microplate 100 is formed from a polymeric material (e.g., polypropylene) and includes a body 106 having formed therein an array of conical or bullet-shaped wells 102 each configured to contain a small sample volume. Polypropylene has few extractables to interfere with the PCR process. The terms “well” and “microwell” may be used interchangeably herein to refer to recesses of a microplate configured to contain limited volumes of materials such as samples.

In accordance with the PCR process, a small quantity of genetic material and a solution of reactants are deposited within each well 102. The microplate 100 is then placed in a thermocycler, which operates to increase and decrease the temperature of the contents within the wells. In an example PCR process, the microplate 100 is placed on a metal heating fixture within the thermocycler. To provide good thermal contact and precise temperature control, the heating fixture is sized and shaped to closely conform to the underside of the microplate 100 and, in particular, to the exterior surface portion of the wells 102. A heated top plate of the thermocycler clamps the microplate onto the heating fixture while the well contents are repeatedly heated and cooled.

Because the microplate 100 is typically made from a non-thermally conductive polymeric material, the walls 105 of the wells 102 are configured to be as thin as possible to enable the thermocycler to effectively heat and cool the well contents. As a result, however, the relatively thin well walls 105 tend to deform in response to the repeated thermal cycling. In addition, the plate body may deform and even thermally degrade. Such degradation may further contribute to warping or twisting of the plate. In order to accommodate the deformation, conventional microplates are formed using relatively non-rigid materials such as polypropylene. Unfortunately, polypropylene tends to strain in response to thermally-induced stress.

As a result of the deformation of the relatively thin well walls 105 and the tendency of the microplate body 106 to change dimensions during thermal cycling, it may be difficult to remove a traditional microplate from the thermocycler. Notably, as the number of wells 102 (and the overall size) of the microplate 100 increases, the force required to remove a deformed microplate 100 from the thermocycler increases, which may cause further damage. Moreover, robotic handling systems may have difficulty manipulating the microplate 100 and removing it from the thermocycler.

Additionally, when microplates are subjected to thermal cycling and/or other analytical or processing steps, such microplates are frequently covered with a cover or sealed with a sealing film to inhibit evaporation of contents within the wells. In certain instances, however, seals may lose or lack contact in one or more locations, thereby subjecting contents of wells to undesirable evaporation.

Accordingly, there is a need for a microplate free of the aforementioned shortcomings.

### BRIEF SUMMARY

In accordance with certain aspects of the present disclosure, a microplate is provided comprising a body having a first surface and an opposing second surface defining a deck, the body including a plurality of wells formed in the deck and extending from the second surface, a frame peripheral to the plurality of wells, and a plurality of reinforcing ribs formed integral to the body and extending from the second surface. Optionally, a stress-relieving slot may be formed in each opposing length of the frame.

In accordance with additional aspects of the present disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. The peripheral deck portion comprises a plurality of locally increased peripheral deck thickness regions formed integral to the body, extending from the second surface, and configured to enhance stiffness of the microplate.

In accordance with additional aspects of the present disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. The peripheral deck portion comprises a plurality of locally increased peripheral deck thickness regions formed integral to the body and comprising a plurality of dots, extending from the second surface, and configured to enhance stiffness of the microplate.

In accordance with additional aspects of the present disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. Additionally, the frame comprises at least one wall arranged non-parallel to the deck, the at least one wall comprises an outer surface and an inner surface, and the inner surface comprises a plurality of locally increased wall thickness regions configured to enhance stiffness of the microplate.

In accordance with additional aspects of the present disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. Each well of the plurality of wells comprises a raised rim including a top surface elevated above the first surface, an intermediate surface arranged between the top surface and the first surface, an inner transition surface extending between the intermediate surface and the top surface, and an outer transition surface extending between the intermediate surface and the top surface. Additionally, an angle between the inner transition surface and the top surface is in a range of from 100 to 170 degrees, and an angle between the outer transition surface and the top surface is in a range of from 100 to 170 degrees.

In accordance with additional aspects of the present disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. Each well of the plurality of wells comprises a raised rim including a top surface elevated above the first surface, and the microplate further comprises at least one elevated sealing feature extending from the first surface, and being arranged between corners of the deck and the plurality of wells.

Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure, and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative, and are not intended to limit the scope of the claims in any manner.

### BRIEF DESCRIPTION OF THE DRAWINGS

The following detailed description of specific embodiments of the present disclosure can be best understood when read in conjunction with the following drawings, where like structure is indicated with like reference numerals and in which:

FIGS. 1A-1C respectively illustrate a perspective view, a cut-away partial perspective view, and a cross-sectional side view of a microplate;

FIG. 2 is a perspective view of an example thermocycler capable of heating and cooling the microplates disclosed herein, together with three cross-sectional views of portions of microplates received by the thermocycler;

FIG. 3 is a bottom plan view of a reinforced microplate according to one embodiment;

FIG. 4A is a cross-sectional view of the reinforced microplate of FIG. 3 taken along section line A-A illustrated in FIG. 3;

FIG. 4B is a cross-sectional view of the reinforced microplate of FIG. 3 taken along section line H-H illustrated in FIG. 3;

FIG. 5A-5B are detailed cross-sectional views of the reinforced microplate of FIG. 3;

FIG. 6 is a detailed top plan view of a corner of the reinforced microplate of FIG. 3;

FIG. 7 is a detailed top plan view of an edge of the reinforced microplate of FIG. 3;

FIG. 8 is a top perspective view of a reinforced microplate including an edge slot according to one embodiment;

FIG. 9 is a bottom perspective view of the reinforced microplate of FIG. 8;

FIG. 10 is an optical micrograph comparing the thermal response for a conventional microplate to a reinforced microplate according to one embodiment;

FIG. 11 is a bottom perspective view of a reinforced microplate including multiple locally increased thickness regions of a deck and a half skirt peripheral frame according to one embodiment;

FIG. 12 is a bottom plan view of the reinforced microplate of FIG. 11;

FIG. 13 is a bottom plan view of a peripheral portion of the reinforced microplate of FIG. 11;

FIG. 14 is a bottom plan view of a corner portion of the reinforced microplate of FIG. 11;

FIG. 15 is a cross-sectional view of the reinforced microplate of FIG. 11 taken along section line N-N illustrated in FIG. 11;

FIG. 16 is a magnified cross-sectional view of a peripheral portion of the microplate of FIG. 15;

FIG. 17 is a cross-sectional view of the reinforced microplate of FIG. 11 taken along section line R-R illustrated in FIG. 11;

FIG. 18A is a cross-sectional view of the reinforced microplate of FIG. 11 taken along section line T-T illustrated in FIG. 11;

FIG. 18B is a magnified cross-sectional view of a peripheral portion of the microplate of FIG. 18A;

FIG. 19 is a magnified cross-sectional view of the peripheral portion of the microplate illustrated in FIG. 18B;

FIG. 20 is a magnified cross-sectional view of a mouth portion of a single well of the microplate illustrated in FIG. 15;

FIG. 21A is a magnified cross-sectional view of a rim portion of the microplate portion illustrated in FIG. 20;

FIG. 21B is a further magnified cross-sectional view of a lip portion of the microplate portion illustrated in FIG. 21A;

FIG. 22 is a bottom perspective view of a reinforced microplate including a full skirt peripheral frame according to one embodiment;

FIG. 23 is a bottom plan view of the reinforced microplate of FIG. 11;

FIG. 24 is a bottom plan view of a corner portion of the reinforced microplate of FIG. 23;

FIG. 25 is a cross-sectional view of the reinforced microplate of FIG. 24 taken along section line AF-AF illustrated in FIG. 24;

FIG. 26 is a magnified cross-sectional view of a peripheral portion of the reinforced microplate illustrated in FIG. 25;

FIG. 27 is a magnified cross-sectional view of a peripheral portion of a raised rim of a well of the reinforced microplate illustrated in FIG. 26;

FIG. 28 is a cross-sectional view of the reinforced microplate of FIG. 24 taken along section line AE-AE illustrated in FIG. 24;

FIG. 29 is a cross-sectional view of a peripheral deck portion of the reinforced microplate illustrated in FIG. 28;

FIG. 30 is a cross-sectional view of an upper central portion of the reinforced microplate illustrated in FIG. 28 showing raised rim portions of two adjacent microwells;

FIG. 31 is an end elevation view of a reinforced microplate including elevated sealing features and a full skirt peripheral frame according to one embodiment, with omission of elevated well rim portions;

FIG. 32A is an end elevation view of a reinforced microplate including elevated sealing features, a full skirt peripheral frame, and elevated well rim portions according to one embodiment;

FIG. 32B is an end elevation view of the reinforced microplate of FIG. 32A arranged proximate to a sealing film prior to application of the sealing film to the reinforced microplate;

FIG. 32C is an end elevation view of the reinforced microplate and sealing film of FIG. 32B following application of the sealing film to the microplate to contact the elevated sealing features and the elevated well rim portions;

FIGS. 33A-33B are bar graphs comparing sealing efficacy (in percent reaction volume loss after thermal cycling) for a film-covered reinforced microplate as disclosed herein versus a conventional film-covered microplate following thermal cycling in an ABI 7900 HT thermocycler and a Maxygene II thermocycler, respectively;

FIGS. 34A-34C are tables providing vertical displacement data for microplate corners relative to a flat surface before and after thermal cycling for a reinforced microplate as disclosed herein and a conventional microplate, for three different thermocyclers, respectively;

FIG. 35A is a table providing vertical distance relative to a center for microplate corners before thermocycling for a reinforced microplate as disclosed herein and four conventional microplates, respectively;

FIG. 35B is a table providing vertical distance relative to a center for microplate corners after thermocycling for a reinforced microplate as disclosed herein and four conventional microplates, respectively;

FIG. 36A is an upper perspective view photograph showing a first conventional Axygen microplate before thermocycling and a second conventional Axygen microplate after thermocycling; and

FIG. 36B is an upper perspective view photograph showing a first reinforced microplate as disclosed herein before thermocycling and a second reinforced microplate as disclosed herein after thermocycling

### DETAILED DESCRIPTION

Reference will now be made in greater detail to various embodiments of the subject matter of the present disclosure, some embodiments of which are illustrated in the accompanying drawings. The same reference numerals will be used throughout the drawings to refer to the same or similar parts.

A microplate comprises a unitary body having reinforcing features that enhance stiffness and minimize deformation of the microplate, especially thermally-induced deformation. In certain embodiments, reinforcing features include a plurality of locally increased thickness regions formed integral to the unitary body of the microplate. Recognizing that injection molding may desirably be used for producing microplates, the use of multiple locally increased thickness regions is preferable over producing large continuous regions of increased thickness, since large continuous regions of increased thickness tend to result in formation of bubbles during the molding process, and such bubbles may lead to manufacturing defects.

In certain embodiments, one or more portions of a deck of a microplate include a plurality of locally increased thickness regions. For example, reinforcing features including locally increased thickness regions are provided on a bottom surface of a microplate, such as along peripheral deck portions laterally surrounding a plurality of wells extending from the deck, and/or between individual wells of the plurality of wells. In certain embodiments, the reinforcing features include ribs or struts that are integrally formed on a bottom surface of the microplate. Further, the frame of the microplate can include one or more slots that disrupt the effects of thermal expansion and limit thermally-induced strain. In embodiments, the microplate is made by injection molding in a 1-shot process and thus comprises a single polymer material. The slots may be formed in situ, i.e., via the molding process. Alternatively, the slots may be formed after molding the microplate such as by cutting the frame.

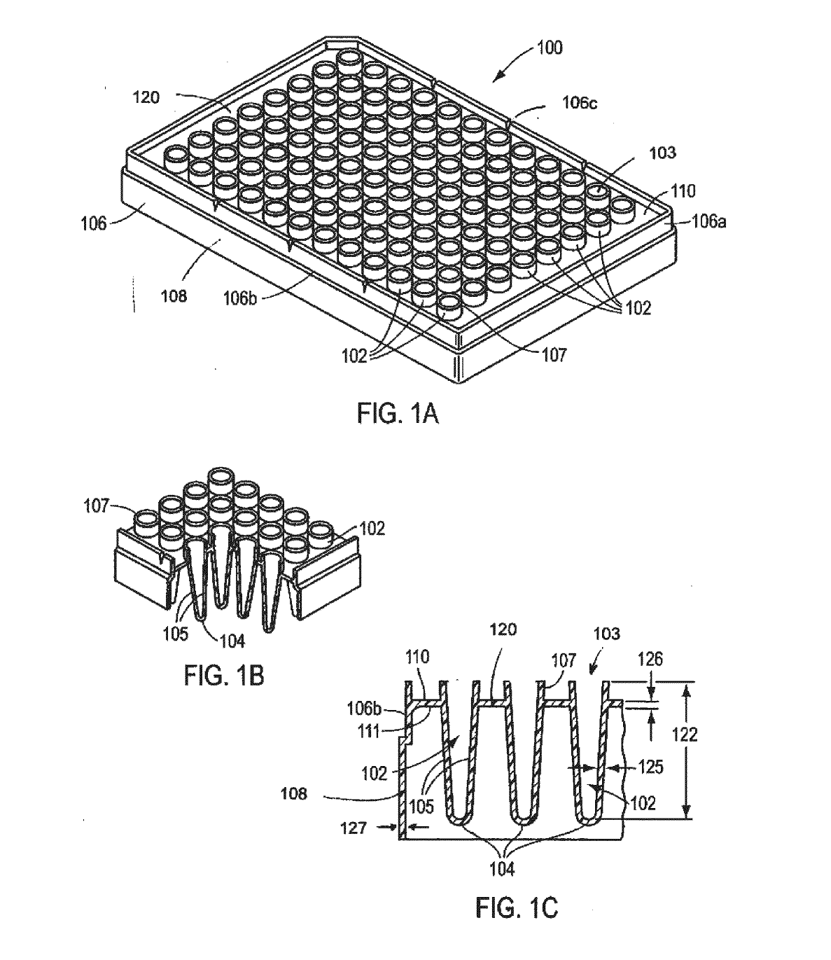

Referring again to FIGS. 1A-1C, there are illustrated different views of an exemplary microplate 100. The microplate 100 includes a body 106 that is manufactured from a polymeric material. Examples of polymeric materials that may be used to produce microplates disclosed herein include polypropylene, polycarbonate, polystyrene, polyvinyl chloride, polyethylene terephthalate, cyclic olefin copolymers, and copolymers or other combinations of the foregoing materials, optionally in conjunction with one or more additional materials. The body 106 as shown has a rectangular shape having major and minor lengths (or a length and a width that is less than the length) and includes a top surface 110 bordered by a frame 108 oriented substantially orthogonal to the top surface 110. The thickness 127 of the frame 108 is defined as the distance between opposing inner and outer surfaces of the frame 108 along a line substantially perpendicular to the opposing surfaces.

Referring to FIG. 1C, the body 106 may have a substantially planar top surface 110 and an opposing bottom surface 111 defining a deck 120 having a thickness 126. The bottom surface 111 may be substantially planar. The thickness 126 of the deck 120 is defined as the distance between the top surface 110 and the bottom surface 111 along a line substantially perpendicular to a major plane of the top surface 110.

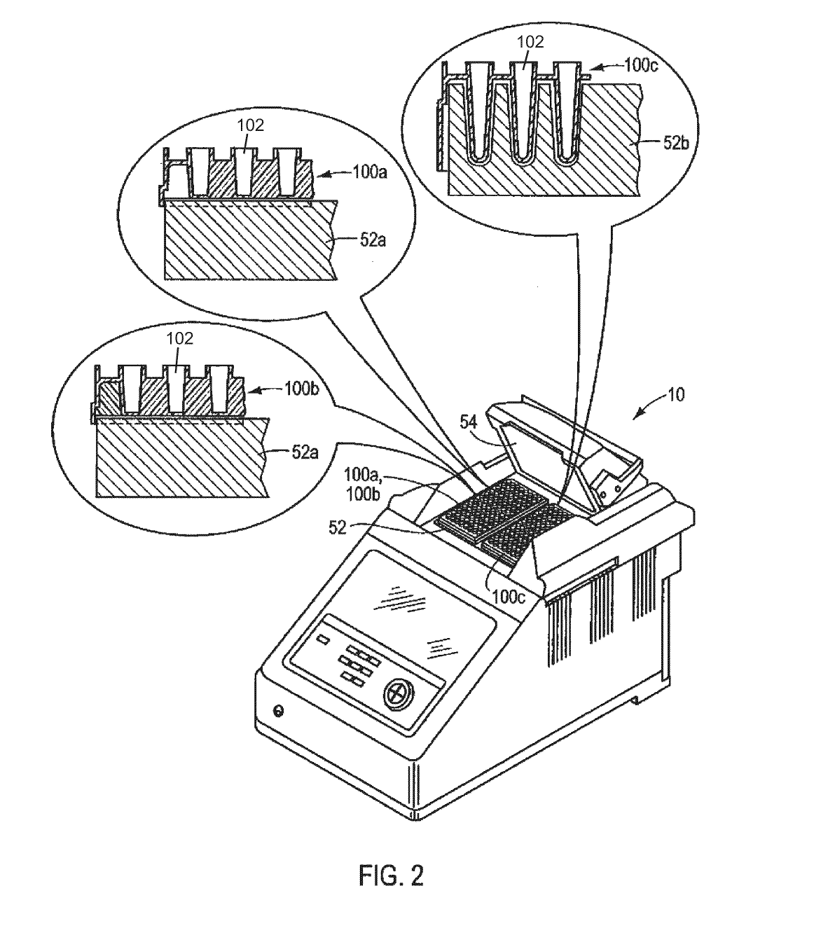

In the illustrated embodiment, the microplate 100 includes an array of ninety-six wells 102 (e.g., arranged in nine rows and twelve columns) formed in the deck 120 and extending downwardly from the bottom surface 111. The microplate 100 is configured to be placed within a thermocycler as described in greater detail below with reference to FIG. 2. It should be understood that the body 106 can be provided in any number of other geometric shapes (e.g., square or triangular) depending, for example, on the desired arrangement of the wells 102 and the design of the thermocycler.

Each well 102 of the plurality of wells 102 includes a well opening 103, an opposing well bottom 104, and a well wall 105 defining a well volume between the opening 103 and the bottom 104. In certain embodiments, raised ridges 107 extend from the top surface 110 peripheral to each well opening 103 to form an elevated rim for each well 102. The ridges 107, if provided, may be used to receive a sealing film (not shown) to form a seal for each well 102 and thereby reduce evaporation of the contents of each well 102 during a thermo cycling process.

A microplate as disclosed herein may include any number of wells, e.g., 2 or more wells, for example 9, 16, 20, 30, 36, 96, 384 or 1536 wells. The wells 102 may be arranged in a closely packed array or in a regular array of rows and columns. In the illustrated embodiments, the wells in each row and/or column are substantially aligned with wells in adjacent rows and/or adjacent columns. In other embodiments, the wells in each row and/or column are offset from wells in adjacent rows and/or adjacent columns. The embodiments illustrated in FIG. 1A-1C, for example, show a microplate 100 comprising plural wells arranged in an 8×12 array with the wells 102 in each row substantially aligned with wells 102 in adjacent rows, and with the wells 102 in each column substantially aligned with wells 102 in adjacent columns.

The well openings 103 can have any suitable geometric shape. Non-limiting examples of suitable shapes for the well openings 103 include polygonal, triangular, quadrangular, rectangular, square, trapezoidal, rhomboidal, pentagonal, hexagonal, heptagonal, octagonal, nonagonal, decagonal, circular, ovoid, ellipsoid, and curvilinear polygonal. In various embodiments, at least one well 102 includes a well opening 103 having a shape in common with at least one other well of the plurality of wells. In some embodiments, each well 102 has a well opening 103 with the same shape as every other well of the plurality of wells. In other embodiments, each well 102 of the plurality of wells has a well opening 103 having a unique shape. In the embodiments illustrated in FIGS. 1A-1C, each well 102 has a well opening 103 that is substantially circular.

Microplate 100 may optionally include a peripheral apron 106a extending upwardly from the top surface 110, generally above and proximate to the downwardly extending frame 108. In some embodiments, at least a portion of the apron 106a extends substantially perpendicular to the top surface 110 of the deck 120. In other embodiments, at least a portion of the apron 106a extends at an angle greater than or less than 90° with respect to the top surface 110 of the deck 120. In some embodiments, the apron 106a can accommodate (e.g., may be configured to receive) a skirt of a microplate cover (not shown). In other embodiments, the microplate 100 does not include an apron 106a.

The apron 106a, if provided, may include an outer rim 106b. In various embodiments, at least a portion of the outer rim 106b extends along a periphery of the free edge of the apron 106a. In other embodiments, the apron 106a does not include an outer rim 106b.

In various embodiments, the microplate 100 may optionally include one or more alignment features for purposes of aligning the microplate body 100 within another device—such as, for example, a gripping device, a handling system, a thermocycler, or a storage device. According to certain embodiments, such alignment features are chosen from cutouts, recesses, protrusions, and combinations thereof. In the embodiments illustrated in FIGS. 1A-1C, for example, the apron 106b includes a plurality of laterally spaced alignment notches 106c.

The wells 102 may have any suitable shape configured to contain a desired fluid volume. In various embodiments, the shape of the wells is defined principally by the well wall 105. Non-limiting examples of well shapes includes conical, frustoconical, rounded conical, right or oblique pyramidal, right or oblique frustopyramidal, cylindrical, cylindrical with a rounded end, right or oblique prism shaped, uniform or nonuniform prism-shaped, bullet-shaped, and combinations thereof.

In various embodiments, at least one well 102 has at least one plane of symmetry. In some embodiments, the at least one plane of symmetry includes a major axis of the well 102. The major axis extends from a center of the well opening 103 to the nadir of the well bottom 104, for example. In some embodiments, at least one well 102 is radially symmetric about the major axis of the well 102. Other embodiments include at least one well 102 that lacks a plane of symmetry. In some embodiments, the well 102 has a cross-section taken along a plane substantially perpendicular to the major axis of the well that is substantially the same shape over the depth of the well 102. In other embodiments, the well 102 has a cross-section taken along a plane substantially perpendicular to the major axis of the well that varies over the depth of the well 102. In some embodiments, as shown in FIGS. 1A and 1B for example, each well 102 has a circular cross-section taken along a plane substantially perpendicular to the major axis of the well 102.

Non-limiting methods for forming the microplate include injection molding, injection compression molding, vacuum formation with a female mold and a male plug assist, and combinations thereof. In embodiments, the microplate is molded from a same material composition (e.g., a single polymer material) in a single (1-shot) molding step such that the microplate body comprises wells and reinforcing members that are formed integral to the body. Such a microplate is free of any over-molded or attached components.

The microplate 100 may comprise a polymeric material. In various embodiments, the polymeric material has at least one characteristic chosen from being biologically inert, being chemically inert, having low biological reactivity, being thermoplastic, being moldable, being re-moldable, having low extractables, being optically transparent, being optically translucent, being transparent to infrared radiation, and being transparent to ultraviolet radiation. In some embodiments, the microplate body 100 is formed from polycarbonate, polystyrene, polypropylene, polyvinyl chloride, polyethylene terephthalate, cyclo-olefins, or combinations thereof, which are thermoplastic, moldable, re-moldable, chemically inert, optically translucent or transparent, and have low extractables.

Many of the foregoing materials may have low working temperatures, however, and thus the microplate 100 if not suitably designed may deform or undergo other undesirable effects during or after the thermocycling process. Deformation may include warping, twisting, or other deviations from the original planar conformation of the top surface 110 of the deck 120.

In addition, as noted previously, some polymeric materials, for example polypropylene, may strain in response to thermally-induced stress. Further, some polymeric materials, including polypropylene, can harbor residual stress from non-uniform cooling following a molding process, for example an injection molding process. The thermally-induced stress and/or the residual stress may result in deformation of the microplate during or after the thermocycling process.

As a result of the deformation of the microplate 100 during thermal cycling, it may be difficult to remove a microplate main body 100 from a thermocycler, as deformation from the original planar conformation can result in changes in the overall dimensions of the microplate 100, which in turn may exceed the tolerances of the microplate in operation. As the number of wells 102 (and the overall size) of the microplate 100 increases, the force required to remove the microplate 100 from the thermocycler may increase, which may further damage the microplate. Moreover, robotic handling systems may have difficulty manipulating and/or removing a deformed microplate from the thermocycler. In addition, the microplate material may thermally degrade as a result of the thermal cycling. Such degradation may further contribute to warping or twisting of the microplate 100.

FIG. 2 is a perspective view of an exemplary thermocycler 10 capable of heating and cooling the well contents of one or more microplates 100a, 100b, 100c, etc. (which may embody reinforced microplates 100a, 100b, 100c as disclosed herein), together with three cross-sectional views of portions of microplates 100a, 100b, 100c received by the thermocycler 10. In accordance with the PCR process, a small quantity of genetic material and a solution of reactants are deposited within one or more microplate wells 102. Optionally, the microplate is covered (e.g., with a cover) or sealed (e.g., with a sealing film) to inhibit the evaporation of the contents within the wells 102. Thereafter, the reinforced microplates 100a, 100b, 100c are placed in the thermocycler 10, which operates to cycle the temperature of (i.e., repeatedly heat and cool) the contents within the wells 102.

As illustrated in FIG. 2, reinforced microplates 100a, 100b are positioned onto a metal heating fixture 52 such as heating fixture 52a in the example of a MJ Alpha-1200 thermocycler. The metal heating fixture 52a can be relatively flat to conform to flat-bottomed wells 102, such as associated with microplates 100a, 100b. In a further embodiment, a reinforced microplate 100c can be positioned onto a metal heating fixture 52b such as (for example) a GeneAmp® PCR System 9700. The metal heating fixture 52b has a series of cavities that are shaped to closely conform to the exterior dimensions of the rounded bottom wells 102 of microplate 100c.

The thermocycler 10 also has a heated top plate 54 (shown in the open position in FIG. 2) that clamps the reinforced microplates 100a, 100b, 100c onto the metal heating fixtures 52a, 52b before the thermocycler 10 repeatedly heats and cools the well contents. For instance, the thermocycler 10 can cycle the temperature of the contents within the wells of the reinforced microplates 100a, 100b, 100c over a temperature range of 25° C. to 95° C. as many as thirty times during the PCR process, which may have duration of up to 4 hours, e.g., 0.5, 1, 2, 3, or 4 hours. During a typical PCR process, the temperature of the top plate 54 is held constant (e.g., 100° C.) to minimize condensation while the temperature of the heating fixture (e.g., fixtures 52a, 52b) is cycled. This temperature differential may exacerbate distortion or warping of the microplates subjected to thermocycling during the PCR process.

According to various disclosed embodiments, the microplates comprise a reinforced structure that may include multiple localized regions of different thicknesses. With reference to FIG. 1C, a microplate comprises a deck 120 having a deck thickness 126, a frame 108 having a frame thickness 127, and a plurality of wells 102, each well having a well wall thickness 125. In embodiments, the frame thickness 127 is greater than or equal to the deck thickness 126, e.g., the frame thickness 127 can be 1, 1.1, 1.2, 1.3, 1.4, 1.5, 2, 2.5 or 3 times the deck thickness 126, including ranges between any of the foregoing. In embodiments, the deck thickness 126 is greater than or equal to the well wall thickness 125, e.g., the deck thickness 126 can be 1, 1.1, 1.2, 1.3, 1.4, 1.5, 2, 2.5 or 3 times the well wall thickness 125, including ranges between any of the foregoing. According to various embodiments of the disclosure, the frame thickness 127 is greater than the deck thickness 126 and the deck thickness 126 is greater than the well wall thickness 125.

Thinner well walls 105 and/or well bottoms 104 may allow for improved thermal conductivity, while a thicker frame 108 may aid in resisting or reducing undesired deformation of the microplate 100. As such, the use of a microplate having regions of different thicknesses may facilitate handling of the microplate by a scientist or robotic handling system, for example to remove the microplate from the thermocycler after completion of a PCR process.

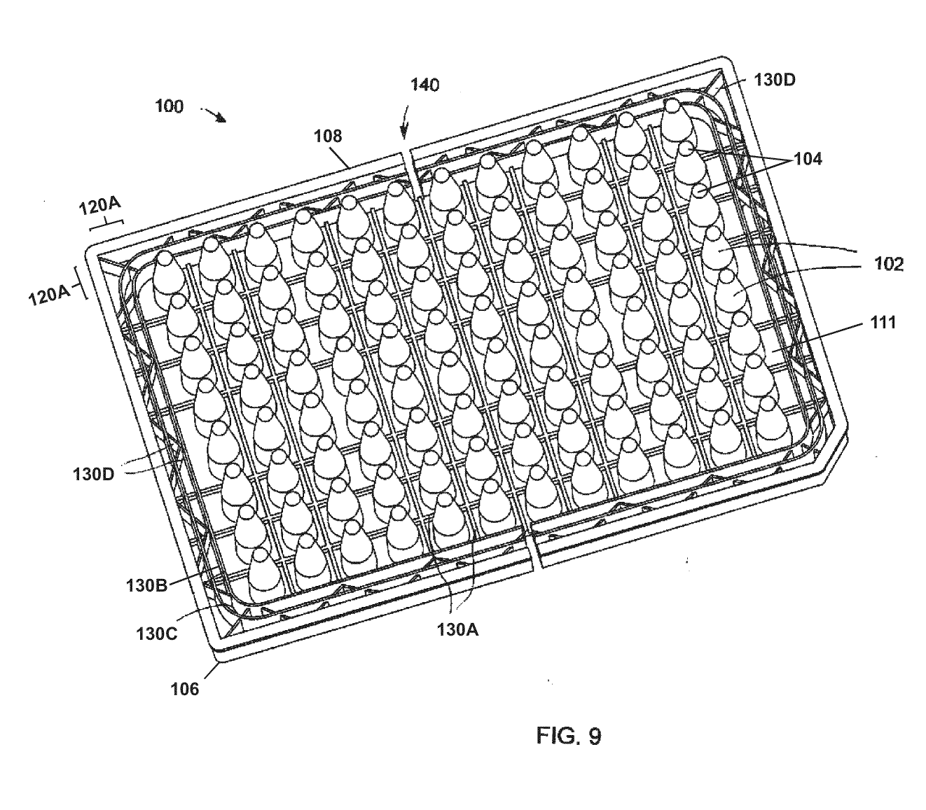

Referring to FIG. 9, the microplate, in embodiments, includes a plurality of reinforcing rib members 130 that project downwardly (e.g., orthogonally) from the plane of the bottom surface 111. The reinforcing rib members 130 may be characterized by a height from the bottom surface 111, a length, and a width (i.e., thickness). In combination, multiple reinforcing rib members embody a plurality of locally increased thickness regions. One or more of the height, length and width of a first rib member may be the equal to or different than one or more of the height, length and width of a second rib member. The height of a reinforcing rib member may range, for example, from 0.02 to 0.25 inches, e.g., 0.02, 0.03, 0.04, 0.05, 0.1, 0.15, 0.2 or 0.25 inches, including ranges between any of the foregoing values. The width of a reinforcing rib member may range, for example, from 0.01 to 0.09 inches, e.g., 0.01, 0.02, 0.03, 0.04, 0.05, 0.06, 0.07, 0.08 and 0.09 inches, including ranges between any of the foregoing values.

Portions of the rib members 130 may intersect with one another to form a corrugated-type reinforcing network at an underside of the microplate 100, e.g., the rib members may be arranged in folds or alternate furrows and ridges. Plural rib members may be disposed on the deck bottom surface peripheral to the well array, thereby embodying a plurality of locally increased peripheral deck thickness regions. Rib members located peripheral to the well array may be arranged with a length that is parallel to, orthogonal to, and/or at an oblique angle to the major or minor lengths (also known as length or width dimensions) of the microplate. For instance, one or more rib members may be configured to form one or more continuous or semi-continuous loops that extend along a periphery of the microplate. Plural loops may be concentric. A semi-continuous loop may be interrupted at one or more points along its length. An example distance from an outer edge of the microplate to a peripheral rib member may range from ⅛ inch to ⅜ inch, for example.

In addition to or in lieu of such an arrangement including locally increased peripheral deck thickness regions that may include ribs peripheral to a well array, locally increased deck thickness regions such as rib members may be disposed between wells, i.e., between the rows and/or columns of wells. Such rib members may be arranged with a length that is parallel to a close packed, row or column direction of the well array, i.e., arranged as substantially linear segments. The rib members essentially form regions of the deck that are locally thicker (i.e., by an amount equal to the height of the rib member).

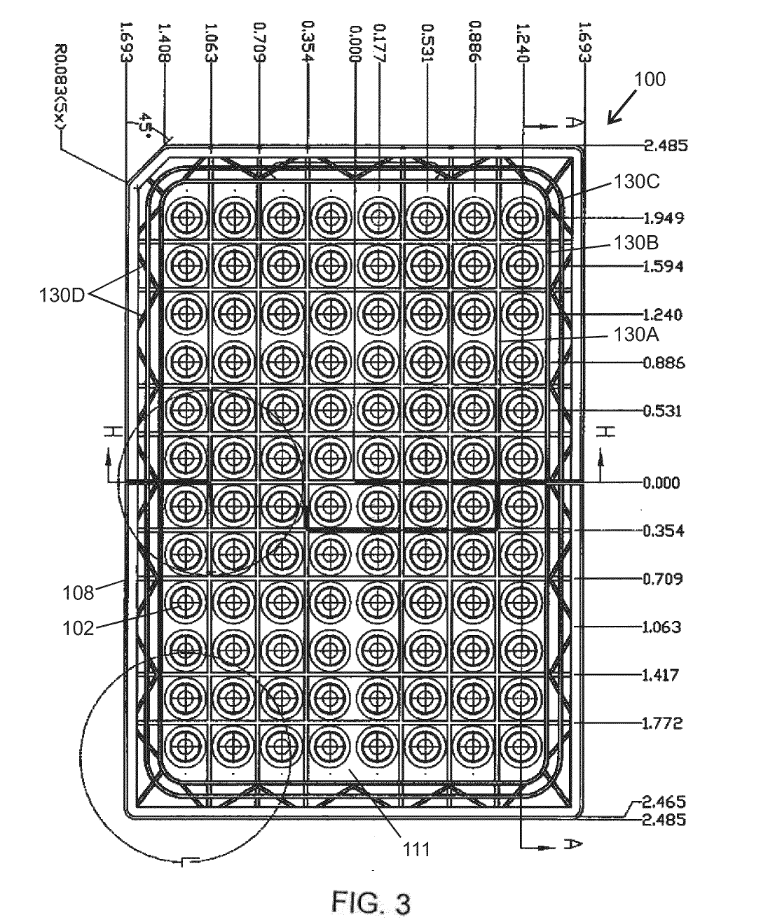

Additional aspects of the reinforced microplates, which include reinforcing ribs, are disclosed herein with reference to the engineering drawings of FIGS. 3-7 where length measurements are given in inches.

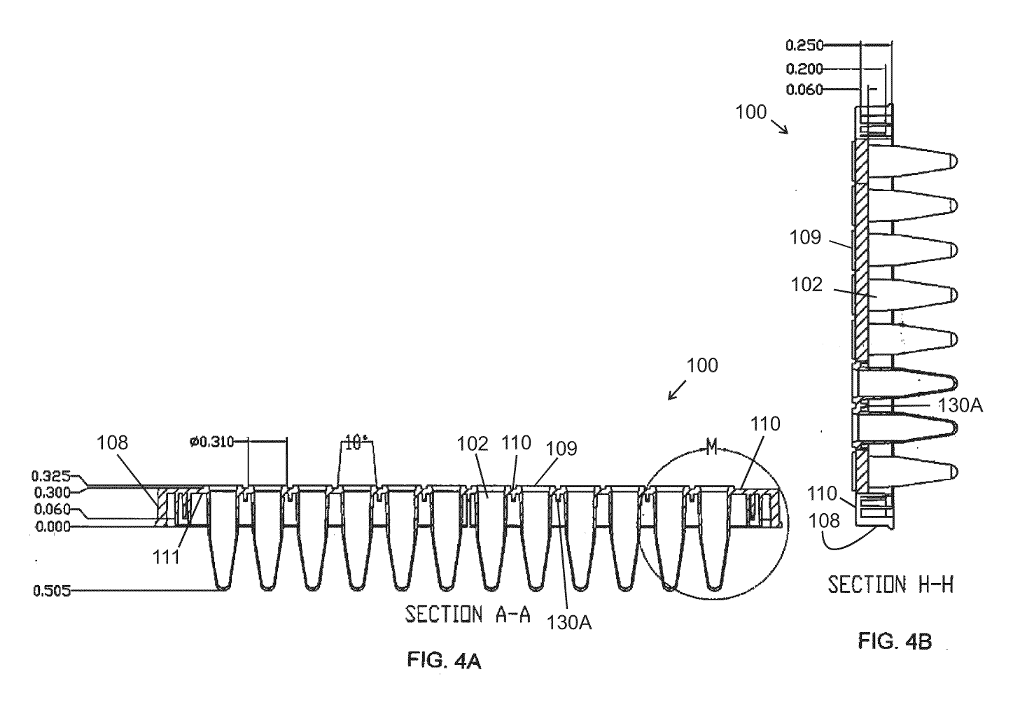

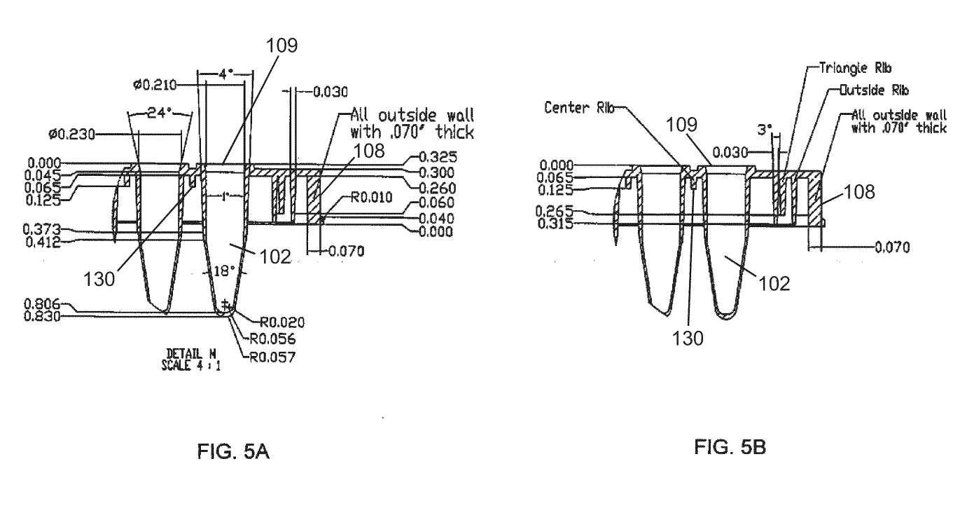

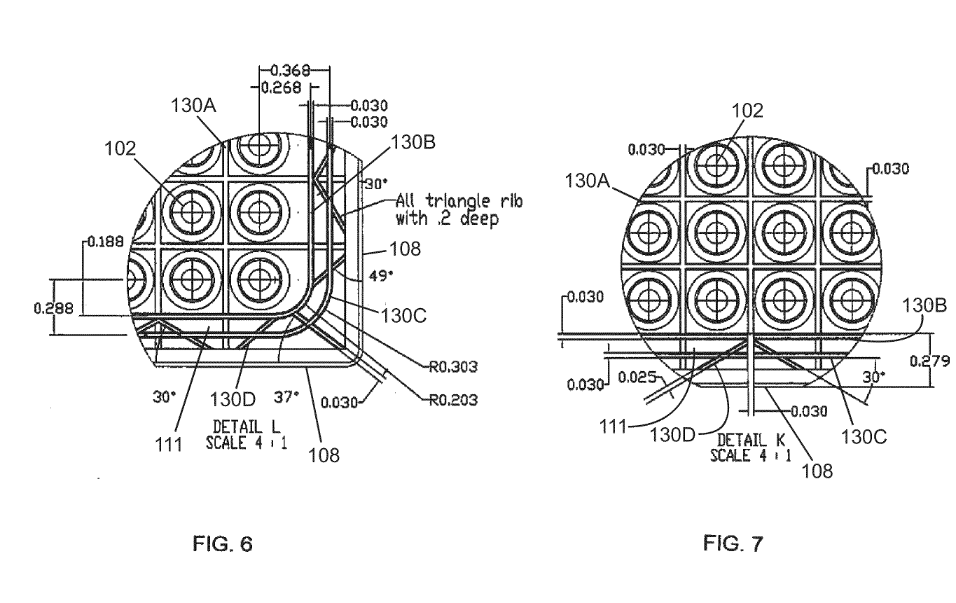

FIG. 3 is a bottom plan view of a reinforced microplate 100 including an array of microwells 102 and multiple locally increased thickness regions 130A-130D extending downward from a bottom surface 111 of a deck that is peripherally surrounded by a downwardly-extending frame 108. The locally increased thickness regions 130A-130D include inter-well rib members 130A as well as multiple increased peripheral deck thickness regions, including a first continuous peripheral rib 130B surrounding the array of microwells 102, a second continuous peripheral rib 130C surrounding the first continuous peripheral rib 130B, and a network of peripheral rib members 130D arranged at various angles. Corresponding cross-sectional views of the microplate 100 taken along section lines A-A and H-H are depicted in FIGS. 4A and 4B, respectively, which depict the frame 108 extending downward approximately half the depth of the microwells 102. FIGS. 4A and 4B further depict the microwells 102 as each having a rim 109 that is raised relative to a top surface 110, and depict inter-well ribs 130 extending downward from a bottom surface 111 of a deck in which the microwells 102 are formed. Detailed views of circular portion M depicted in FIG. 4A are shown in FIGS. 5A and 5B, showing wells 102 each including an elevated rim 109, with downwardly-extending ribs 130 disposed between wells 102, and a deck peripherally surrounded by the frame 108. Detailed views of circular portions L and K depicted in FIG. 3 are shown in FIGS. 6 and 7, showing microwells 102 and locally increased thickness regions 130A-130D embodied in ribs extending downward from the bottom surface 111 of a deck.

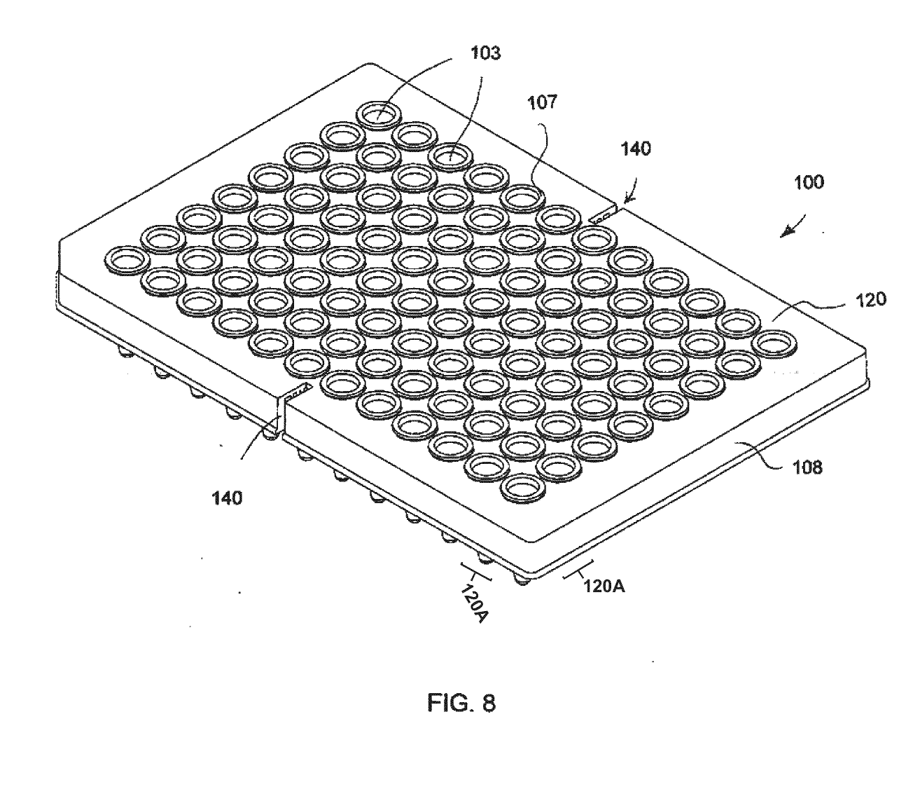

In embodiments, the microplate frame 108 includes one or more slots 140 that cut completely through the frame 108. In embodiments, the one or more slots cut completely through peripheral rib members 130. By way of example, and with reference to FIGS. 8 and 9, which are respectively top and bottom perspective views, a reinforced microplate 100 can include a pair of slots 140 each formed in an opposing relationship in a respective major length of the frame 108, such as at a midpoint of the major length. In certain embodiments, each slot 140 is defined at least in the frame 108 and extends in a direction substantially perpendicular to the deck 120. In certain embodiments, the microplate 100 includes a width and a length exceeding the width, wherein the at least one stress-relieving slot 140 is further defined in a peripheral deck portion (i.e., a portion of the deck 120 that surrounds the microwells 102) and extends substantially perpendicular to a lengthwise direction of the microplate 100.

The incorporation of the slots 140 into the frame facilitates the release of stress in the microplate 100, particularly in a microplate exhibiting a complicated stress state due to differences in, inter alia, the shape and thickness of the part, as well as local temperature differences. Without wishing to be bound by theory, the slots 140 permit thermal expansion within the frame 108 without the accumulation of stress that could otherwise deform the microplate 100.

With continued reference to FIGS. 8 and 9, the microplate 100 includes a body 106 defining a deck 120 and an array of microwells 102 each includes a well opening 103 and an opposing well bottom 104. Each microwell 102 includes raised ridges 107 extending upward from the deck 120 proximate to the well opening 103. The deck 120 includes peripheral deck regions 120A arranged between the array of microwells 102 and the frame 108. The microplate further includes locally increased thickness regions 130A-130D embodied in ribs extending downward from the bottom surface 111 of the deck 120.

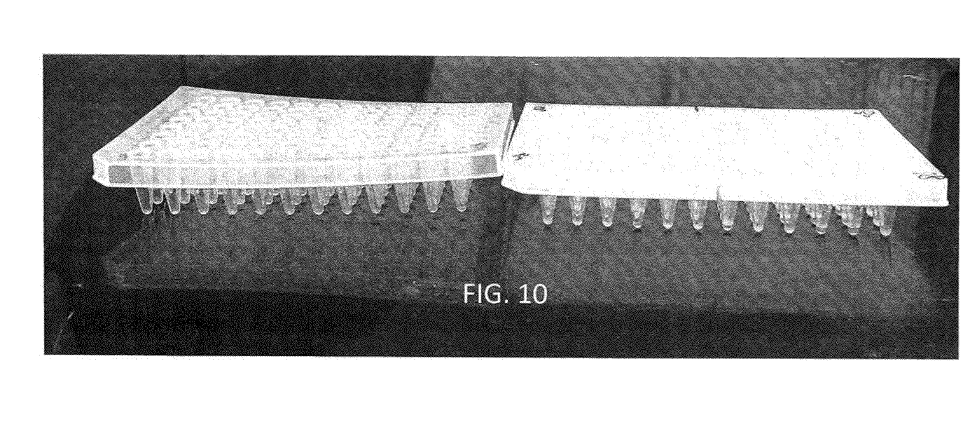

Testing in a thermocycler has shown that the deformation of a reinforced polypropylene microplate as disclosed herein is reduced by 80% in comparison to a conventional (non-reinforced) polypropylene microplate. FIG. 10 is an optical micrograph comparing the thermal cycling response of a conventional polypropylene microplate (at left) to a reinforced polypropylene microplate (at right). Warping is clearly evident in the conventional polypropylene microplate, in contrast to the reinforced microplate.

The use of a reinforced microplate having a rigid structure makes it easy for a scientist or robot handling system to remove the microplate from the thermocycler after completion of the PCR process. This is a marked improvement over the traditional microplate that has a tendency to deform and/or adhere to surfaces of a thermocycler, such as the metal heating fixtures 52a/52b shown in FIG. 2.

Although a reinforced microplate disclosed herein is described as being used in a PCR process, it should be understood that the reinforced microplate can be used in a wide variety of processes. A reinforced PCR microplate may be non-skirted, semi-skirted, or a full-skirted microplate.

In embodiments, the microplate 100 is formed from a transparent material. As used herein, “transparent” means at least 60% transparency (e.g., at least 60, 65, 70, 75, 80, 82, 84, 86, 88, 90, 92, 94, 96, 98, 99 or 100% transparency) for a given wavelength or over a range of wavelengths. In embodiments, for example, the well walls are transparent to visible light (i.e., over the wavelength range of 390 to 700 nm). In embodiments, the well walls are transparent to ultraviolet and/or near-infrared radiation (i.e., over the respective wavelength ranges of 100 to <390 nm and >700 to 2500 nm).

In embodiments, the well walls are characterized by low background fluorescence. Fluorescence is a form of absorbed energy that is reradiated at a lower energy, often as light. The amount of fluorescence (or lack thereof) from reinforced microplates is a key factor in their implementation with, for example, analytical spectroscopy, polarization and imaging, including point-of-care (POC) in vitro diagnostic tests, and other life-sciences analytics such as cellular flow cytometry.

Disclosed herein is a reinforced microplate such as a PCR plate. The entire reinforced microplate may be formed from a same material composition (e.g., a single polymer material) in a single (1-shot) molding process. As such, the microplate is readily recyclable and less expensive to manufacture than comparative plates formed in plural steps and/or which include plural polymer materials arranged in different regions. A reinforced microplate according to certain embodiments includes multiple locally increased thickness regions, such as may be embodied in reinforcing ribs that are incorporated onto the bottom surface of the microplate deck. Stress-relieving slots may be formed in the frame, and optionally in a peripheral deck portion, of a reinforced microplate.

Additional combinations of reinforcing and/or sealing enhancing features may be provided in microplates according to further embodiments.

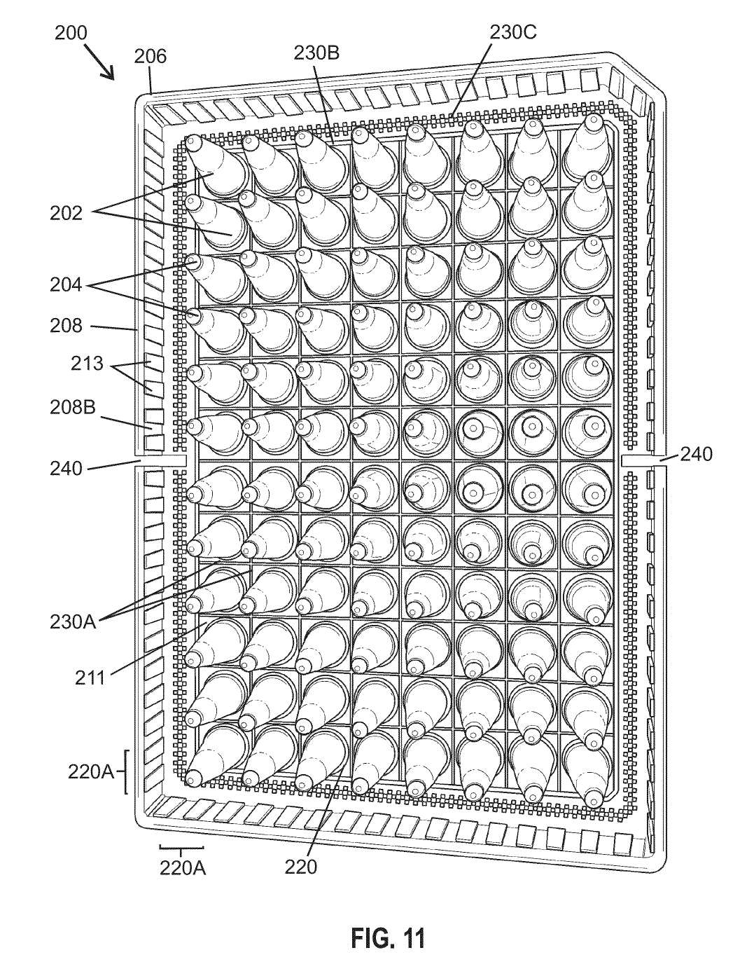

FIG. 11 is a bottom perspective view of a reinforced microplate 200 according to one embodiment, including a body 206 defining a deck 220 and a half-skirt-type peripheral frame 208 each including locally increased thickness regions configured to enhance stiffness of the microplate 200. An array of wells 202 are defined in and extend downward from the deck 220, with each well 202 terminating at a well bottom 204. A plurality of ribs 230A is formed integral to the deck 220, wherein the ribs 230A extend downward from a bottom surface 211, and are configured as a grid between different wells 202 of the array of wells 202. The deck 220 includes a peripheral deck region 220A arranged between the array of wells 202 and the frame 208. Within the peripheral deck region 220A, a substantially rectangular continuous rib 230B serving as a locally increased thickness region is formed integral to the deck 220, extends downward from the bottom surface 211, and surrounds the array of wells 202. Further within the peripheral deck region 220A and surrounding the continuous rib 230B is provided a plurality of locally increased peripheral deck thickness regions embodied in a plurality of dots 230C. As illustrated, the dots 230C are provided in three adjacent rows, and each dot 230C of the plurality of dots 230 comprises a substantially square cross-sectional shape taken along a plane parallel to the deck 220. In certain embodiments, square shaped dots 230C may include a width of from about 0.02 to 0.1 inch, or more preferably in a range of from about 0.025 inch to about 0.05 inch. The square shaped dots 230C may further taper downward to form a truncated obelisk shape.

In certain embodiments, at least a portion of each dot of a subset of dots is arranged in contact with another dot, such as by providing corners of different dots in contact with one another. Although various embodiments disclosed herein include square-shaped dots, it is to be appreciated that dots of any suitable shapes, sizes, patterns, and orientations may be provided. In certain embodiments, multiple dots of different shapes, different sizes, and/or different orientations may be provided in adjacent or non-adjacent regions of the same microplate.

With continued reference to FIG. 11, the frame 208 is arranged along the perimeter of the deck 220, and includes wall arranged non-parallel (e.g., substantially orthogonal) to the deck 220, wherein an inner surface 208B of the wall comprises a plurality of locally increased wall thickness regions embodied in ribs 213 configured to enhance stiffness of the microplate 200. Each rib 213 includes a length extending in a generally vertical direction, and includes a width that tapers with increasing distance away from the bottom surface 211 of the deck 220. The microplate 200 further includes stress-relieving slots 240 extending through the frame 240, wherein at least a portion of each slot 240 extends in a direction substantially perpendicular to the deck 220. As shown, each slot 240 is further defined in the peripheral deck portion 220A and extends substantially perpendicular to a lengthwise direction of the microplate 200.

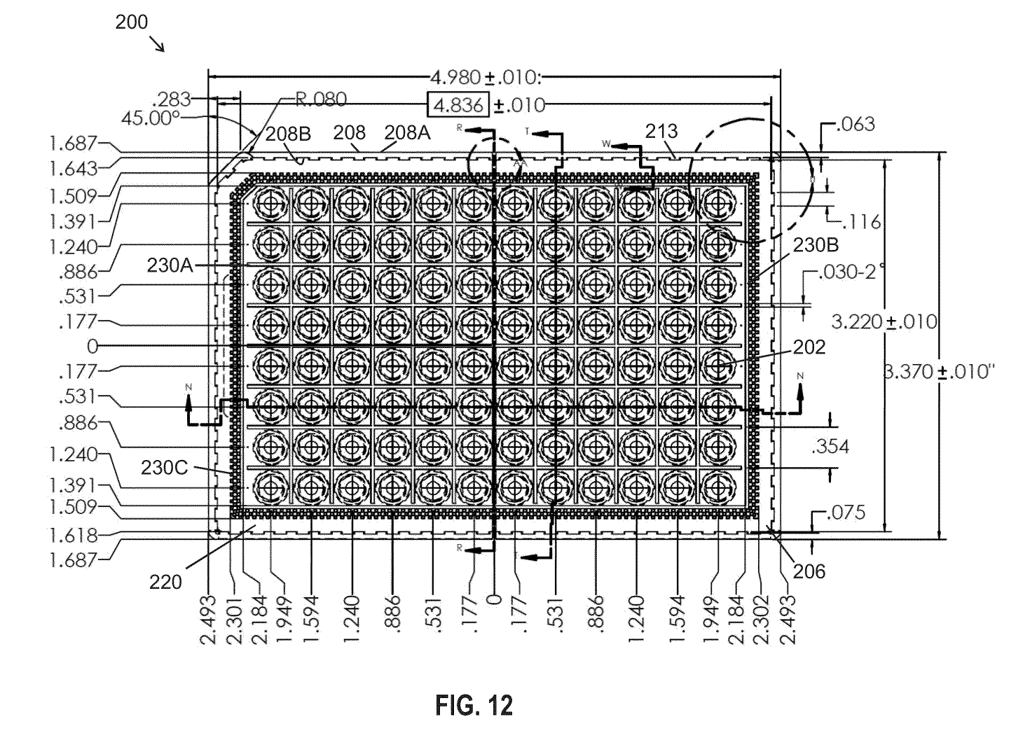

Additional features and aspects of the reinforced microplate of FIG. 11 are disclosed herein with reference to the engineering drawings of FIGS. 12-21 where length measurements are given in inches.

FIG. 12 is a bottom plan view of the reinforced microplate of FIG. 11, showing the body 206 as having a substantially rectangular shape bordered by a frame 208 having an outer surface 208A and an inner surface 208B, with multiple laterally spaced, vertically extending ribs 213 provided along the inner surface 208B. The frame 208 surrounds a deck 220 defining an array of wells 202. Multiple locally increased thickness regions 230A-230C extend from a lower surface of the deck, including inter-well reinforcing rib members 230A as well as multiple increased peripheral deck thickness regions, with the increased peripheral deck thickness regions including a continuous peripheral rib 230B surrounding the array of microwells 202, and plurality of dots 230C surrounding the continuous peripheral rib 230B.

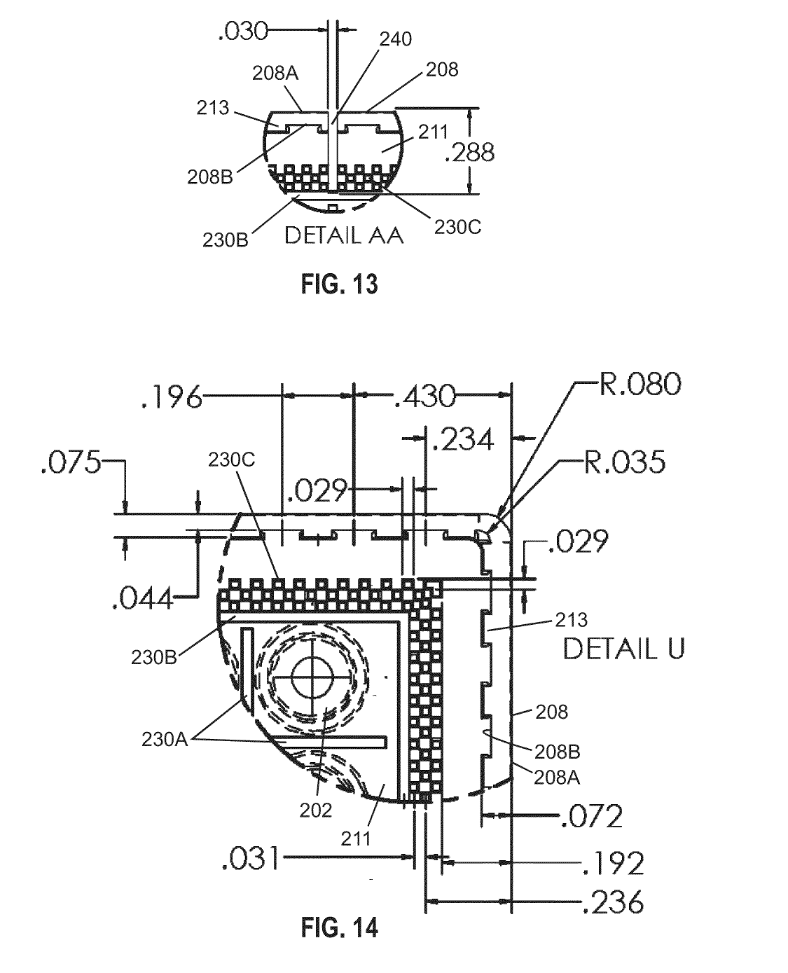

FIG. 13 is a bottom plan view of a peripheral portion of the reinforced microplate of FIG. 11 taken along circular portion AA depicted in FIG. 11. FIG. 13 depicts a portion of the frame 208, including the outer surface 208A and the inner surface 208B, with vertically extending ribs 213 provided along the inner surface 208B. Further depicted are the continuous rib 230B and dots 230C formed on the bottom surface 211, and a slot 240 defined in the frame 208 as well as through a portion of the deck defining the bottom surface 211.

FIG. 14 is a bottom plan view of a corner portion of the reinforced microplate of FIG. 11 taken along circular portion U depicted in FIG. 11. FIG. 14 depicts a corner portion of the frame 208, including the outer surface 208A and the inner surface 208B, with vertically extending ribs 213 provided along the inner surface 208B. Further depicted are inter-well ribs 230A, continuous rib 230B, and dots 230C formed on the bottom surface 211, as well as a well 202.

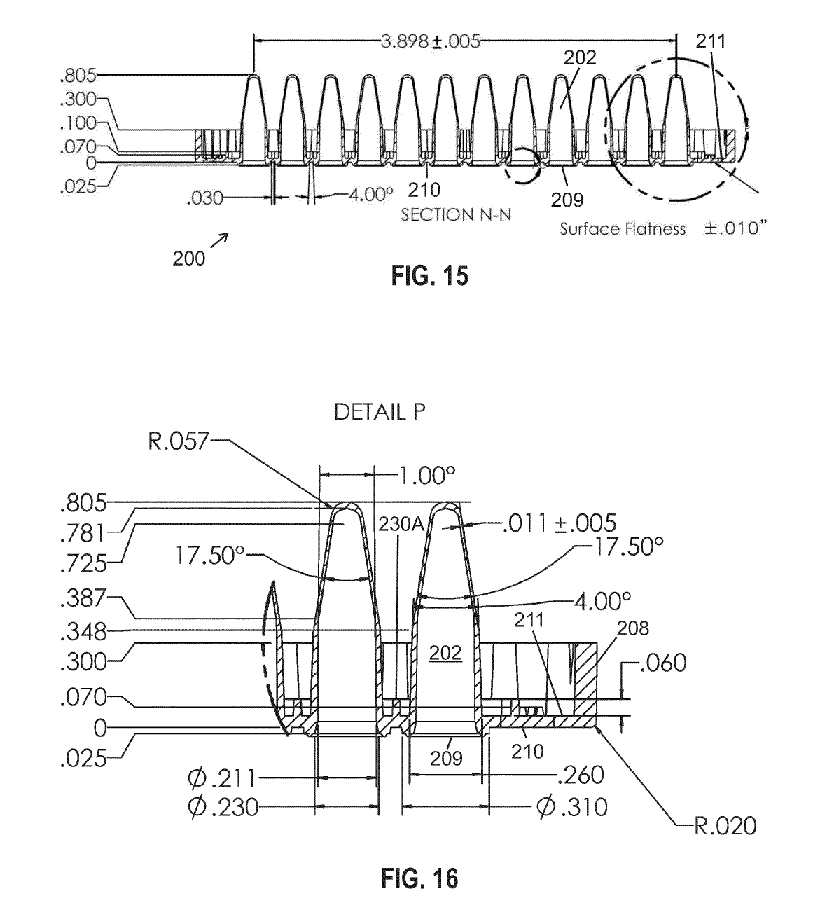

FIG. 15 is a cross-sectional view of the reinforced microplate 200 of FIG. 11 taken along section line N-N illustrated in FIG. 11, and FIG. 16 is a magnified cross-sectional view of a peripheral portion of the microplate of FIG. 15. As illustrated, an array of wells 202 is defined in a deck that is peripherally bounded by a frame 208. Each well 202 includes a rim 209 that is raised relative to a top surface 210 of the deck, and inter-well ribs 230 extend downward from a bottom surface 211 of the deck.

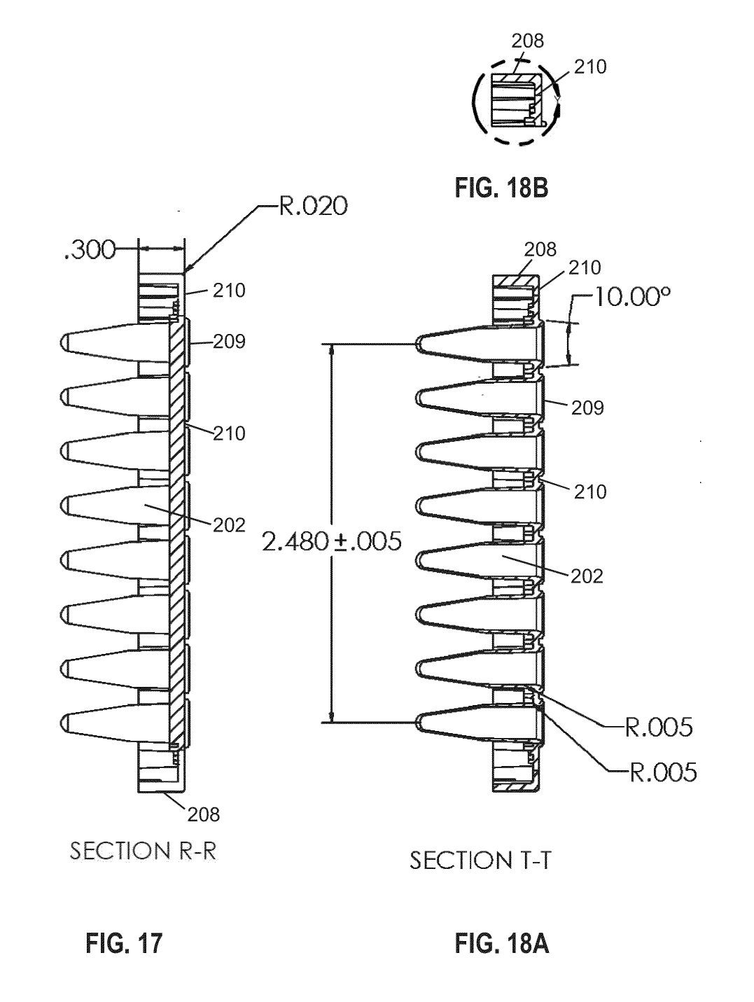

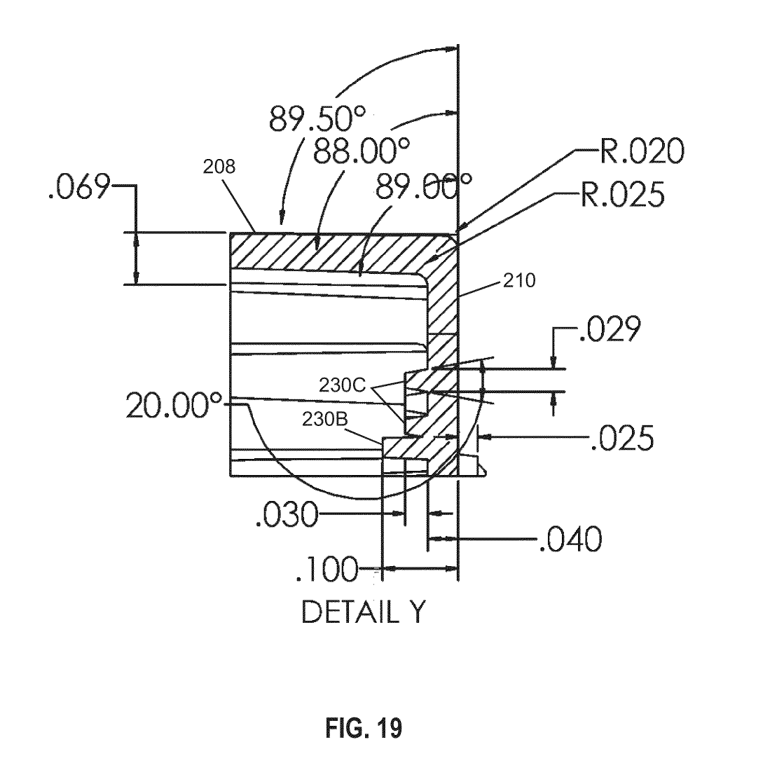

FIG. 17 and FIG. 18A are cross-sectional views of the reinforced microplate of FIG. 11 taken along section lines R-R and T-T, respectively, illustrated in FIG. 11. As illustrated, an array of wells 202 is defined in a deck that is peripherally bounded by a frame 208. Each well 202 includes a rim 209 that is raised relative to a top surface 210 of the deck. FIG. 18B is a magnified cross-sectional view of a peripheral portion of the microplate of FIG. 18A, showing portions of the frame 208 and the top surface 210. FIG. 19 is a magnified cross-sectional view of the peripheral portion of the microplate illustrated in FIG. 18B, showing portions of the frame 208 and the top surface 210 as well as the continuous rib 230B and square-shaped dots 230C.

While various features described hereinabove are directed to providing enhanced structural reinforcement to microplates, further described herein are features intended to enhance sealing of a microplate in operation, which may be beneficial to reduce evaporative loss of contents of microwells during thermocycling and other processing steps.

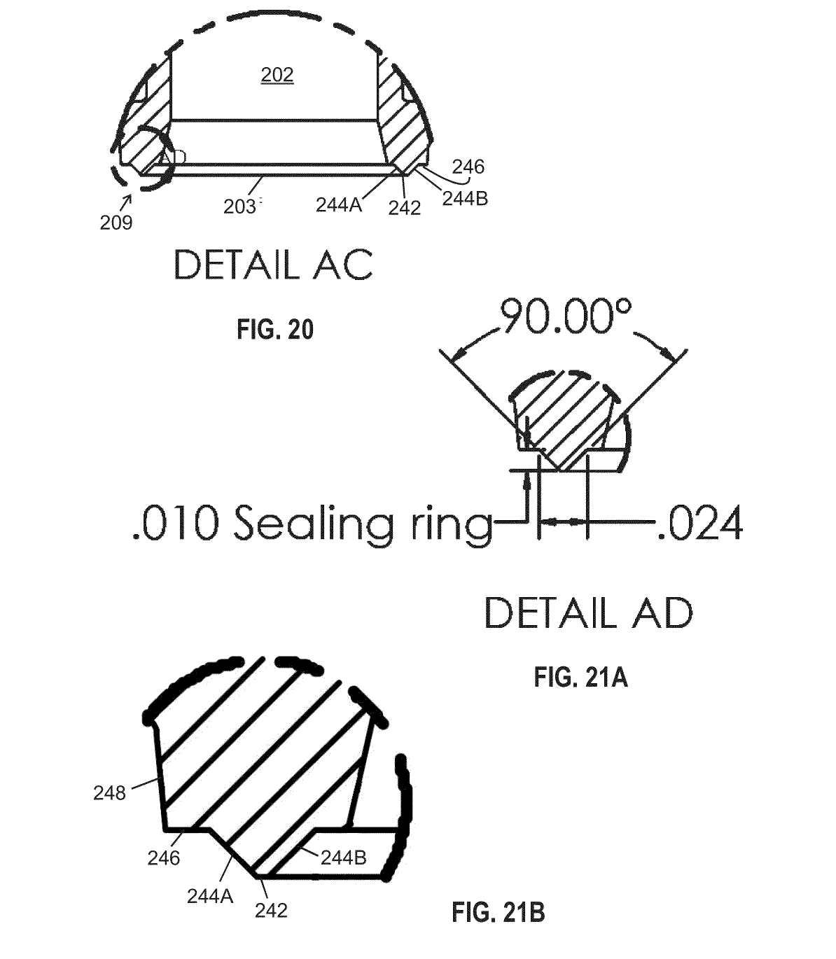

FIG. 20 is a magnified cross-sectional view of a mouth portion of a single well 202 of the microplate illustrated in FIG. 15, while FIGS. 21A and 21B provide a magnified cross-sectional views of a rim portion of the microplate portion illustrated in FIG. 20. As shown in FIGS. 20 to 21B, a microwell 202 includes a well opening 103 that is bounded by a raised rim 209. In certain embodiments, the raised rim 209 is circular in shape; however, other shapes may be provided. The raised rim includes a top surface 242 that is elevated above a first (e.g., top) surface of a deck (not shown), includes an intermediate surface 246, and includes inner and outer transition surfaces 244A, 244B extending between the intermediate surface 246 and the top surface 242. The intermediate surface 246 is adjacent to a riser surface 248 that serves to elevate the raised rim 209 relative to the first or top surface (not shown) of the microplate. In certain embodiments, the top surface 242 may be elevated above the intermediate surface 246 by a distance of from 0.005 to about 0.025 inch, more preferably about 0.01 inch. Preferably, an angle between the inner transition surface 244A and the top surface 242 is in a range of from 100 to 170 degrees, and an angle between the outer transition surface 244B and the top surface 242 is in a range of from 100 to 170 degrees. In certain embodiments, subranges of from 110 to 160 degrees, or from 120 to 150 degrees, or 130 to 140 degrees may be used. As shown in FIG. 21A, an angle between the inner transition surface 244A and the outer transition surface 244B may be about 90 degrees. The top surface 242 serves as a sealing ring for receiving a sealing film or cover (not shown). By providing a raised rim 209 with a top surface 242 and angled transition surfaces 244A, 244B, increased surface area is provided for the raised rim 209 to receive a sealing film and/or sealing cover, thereby enhancing sealing efficacy. Additionally, the narrow width of the top surface 242 may permit localized deformation of a sealing film and/or sealing cover when applied to the microplate, thereby enhancing sealing efficacy.

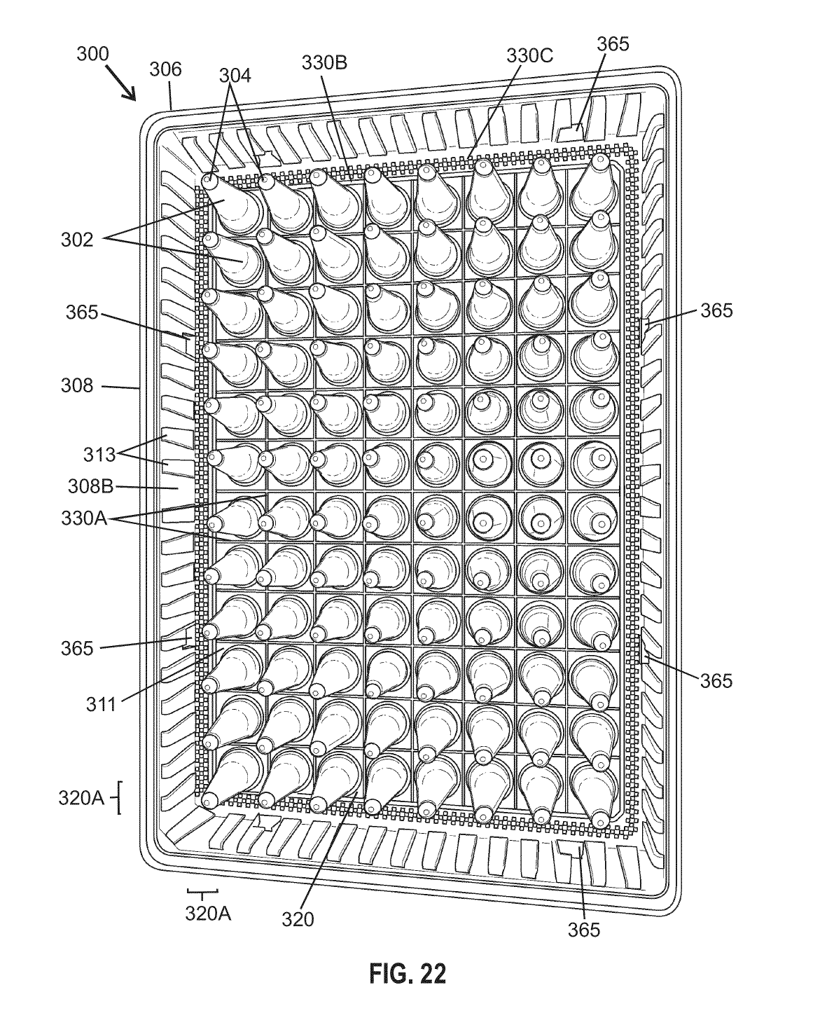

FIG. 22 is a bottom perspective view of a reinforced microplate 300 according to another embodiment, including a body 306 defining a deck 320 and a full-skirt-type peripheral frame 308 each including locally increased thickness regions configured to enhance stiffness of the microplate 300. An array of wells 302 is defined in and extends downward from the deck 320, with each well 302 terminating at a well bottom 304. A plurality of ribs 330A is formed integral to the deck 320, extend downward from a bottom surface 311, with the ribs 330A being configured as a grid between different wells 302 of the array of wells 302. The deck 320 includes a peripheral deck region 320A arranged between the array of wells 302 and the frame 308. Within the peripheral deck region 320A, a substantially rectangular continuous rib 330B serving as a locally increased thickness region is formed integral to the deck 320, extends downward from the bottom surface 311, and surrounds the array of wells 302. Further within the peripheral deck region 320A and surrounding the continuous rib 330B is provided a plurality of locally increased peripheral deck thickness regions embodied in a plurality of dots 330C. As illustrated, the dots 330 are provided in three adjacent rows, and each dot 330 of the plurality of dots 330 comprises a substantially square cross-sectional shape taken along a plane parallel to the deck 320.

With continued reference to FIG. 22, the frame 308 is arranged along the perimeter of the deck 320, and includes a wall arranged non-parallel (e.g., substantially orthogonal) to the deck 320, wherein an inner surface 308B of the wall comprises a plurality of locally increased wall thickness regions embodied in ribs 313 configured to enhance stiffness of the microplate 300. Each rib 313 includes a length extending in a generally vertical direction, and includes a width that tapers with increasing distance away from the bottom surface 311 of the deck 320. The microplate 300 further includes apertures 305 defined in the frame 340 to serve as alignment and/or handling features useful for interfacing the microplate 300 with another device, such as (for example) a gripping device, a handling system, a thermocycler, or a storage device.

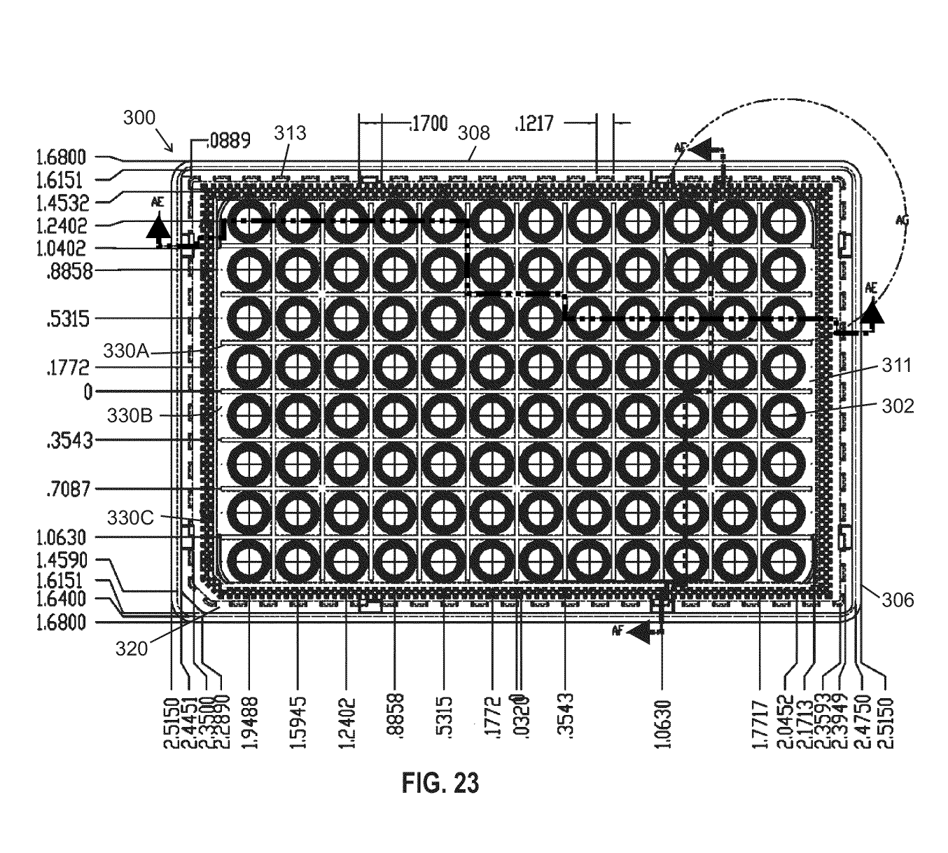

Additional features and aspects of the reinforced microplate of FIG. 22 are disclosed herein with reference to the engineering drawings of FIGS. 23-30 where length measurements are given in inches.

FIG. 23 is a bottom plan view of the reinforced microplate of FIG. 22, showing the body 306 as having a substantially rectangular shape bordered by a frame 308, with multiple laterally spaced, vertically extending ribs 313 provided along an inner surface of the frame 308. The frame 308 surrounds a deck 320 defining an array of wells 302. Further depicted are inter-well ribs 330A, continuous rib 330B, and dots 330C formed on the bottom surface 311, as well as wells 202.

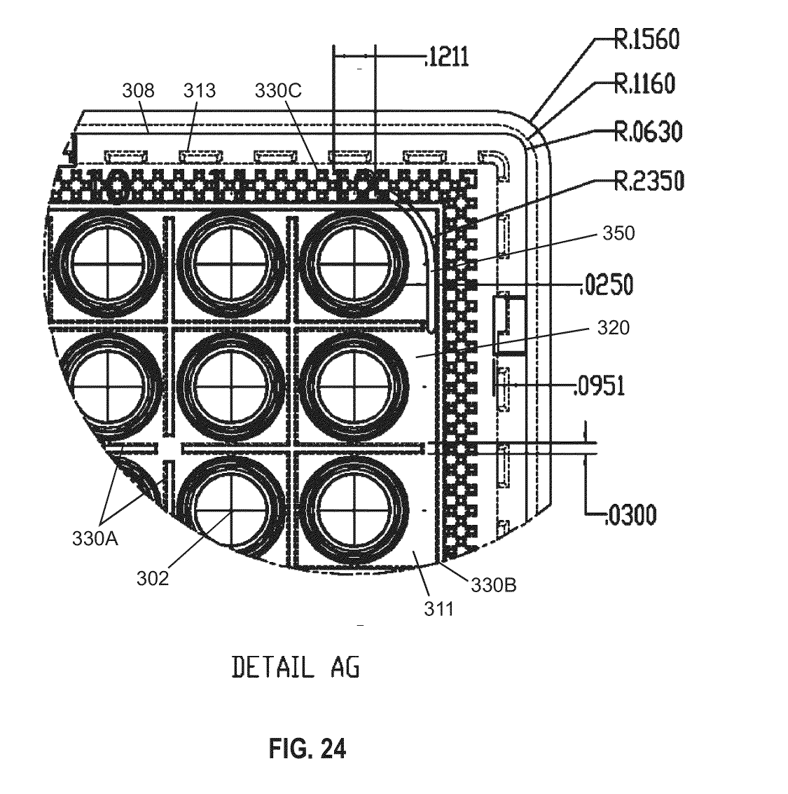

FIG. 24 is a bottom plan view of a corner portion of the reinforced microplate of FIG. 22 taken along circular portion AG depicted in FIG. 22. FIG. 24 depicts a corner portion of the frame 308 with vertically extending ribs 313 provided along an inner surface thereof. Further depicted are inter-well ribs 330A, continuous rib 330B, and dots 330C formed on the bottom surface 311, as well as multiple wells 302. Additionally, FIG. 24 illustrates an elevated sealing feature 350 that has an arc-like curvilinear shape and extends upward from a top surface of the deck 320. In certain embodiments, one or more elevated sealing features 350 may be provided, and may be arranged between corners of the deck 320 and the plurality of wells 302. In certain embodiments, each well 302 includes a raised rim, and preferably the at least one elevated sealing feature 350 has the same or substantially the same height as a top surface of the raised rim of each well 302.

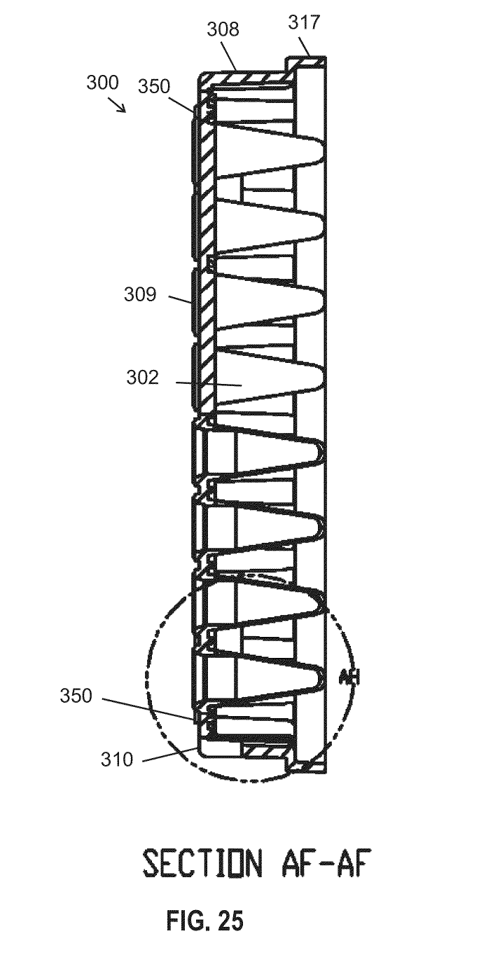

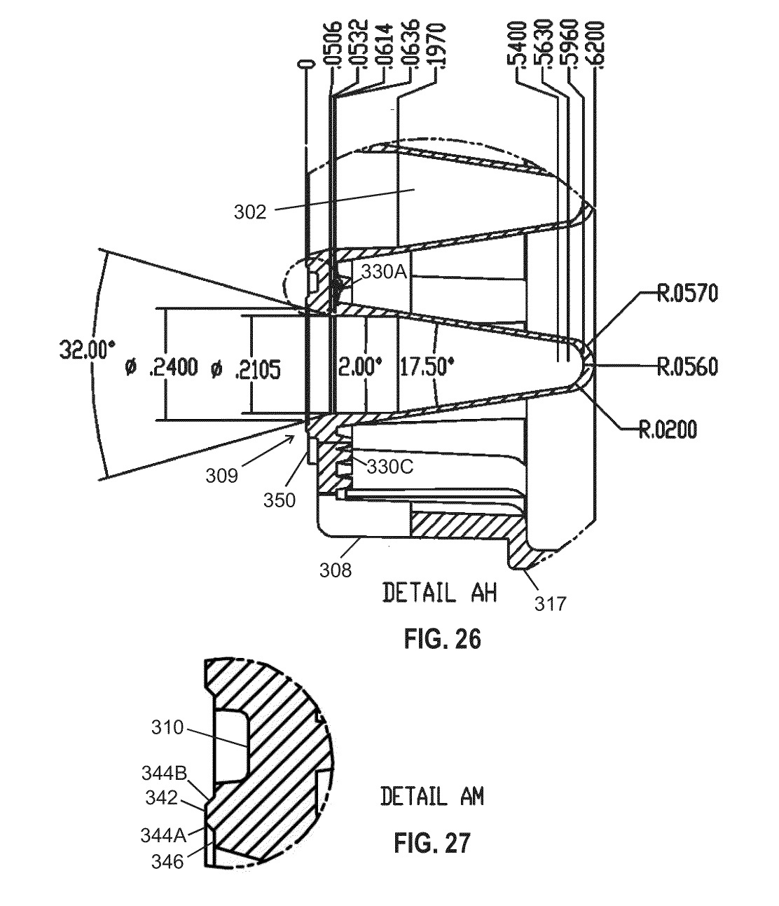

FIG. 25 is a cross-sectional view of the reinforced microplate 300 of FIG. 24 taken along section line AF-AF illustrated in FIG. 24, and FIG. 26 is a magnified cross-sectional view of a circular portion AH of the reinforced microplate illustrated in FIG. 25. Referring to FIG. 25, an array of wells 302 are defined in a deck that is peripherally bounded by a frame 308 that includes a peripheral skirt 317. Each well 302 includes a rim 309 that is raised relative to a top surface 310 of the deck, and elevated sealing features 350 are provided between corners of the microplate 300 and the array of wells 302. Referring to FIG. 26, each well 302 includes a raised rim 309, and a well 302 proximate to a corner of the microplate 300 is adjacent to an elevated sealing feature 350. Downwardly extending ribs 320A are provided in a grid between wells 302, and a plurality of downwardly extending square-shaped dots 320C are provided in a peripheral region of the deck between the wells 302 and the frame 308.

FIG. 27 is a magnified cross-sectional view of a peripheral portion of a raised rim of a well of the reinforced microplate of FIG. 26. As shown in FIG. 27, the raised rim includes a top surface 242 that is elevated above a first (e.g., top) surface of a deck (not shown), includes an intermediate surface 346, and includes inner and outer transition surfaces 344A, 344B extending between the intermediate surface 346 and the top surface 342. In certain embodiments, the top surface 342 may be elevated above the intermediate surface 346 by a distance of from 0.005 to about 0.025 inch, more preferably about 0.01 inch. Preferably, an angle between the inner transition surface 344A and the top surface 342 is in a range of from 100 to 170 degrees, and an angle between the outer transition surface 244B and the top surface 242 is in a range of from 100 to 170 degrees. In certain embodiments, subranges of from 110 to 160 degrees, or from 120 to 150 degrees, or 130 to 140 degrees may be used. The top surface 342 serves as a sealing ring for receiving a sealing film or cover (not shown). By providing a raised rim with a top surface 342 and angled transition surfaces 344A, 344B, increased surface area is provided for the raised rim 309 to receive a sealing film and/or sealing cover, thereby enhancing sealing efficacy. Additionally, the narrow width of the top surface 342 may permit localized deformation of a sealing film and/or sealing cover when applied to the microplate, thereby enhancing sealing efficacy.

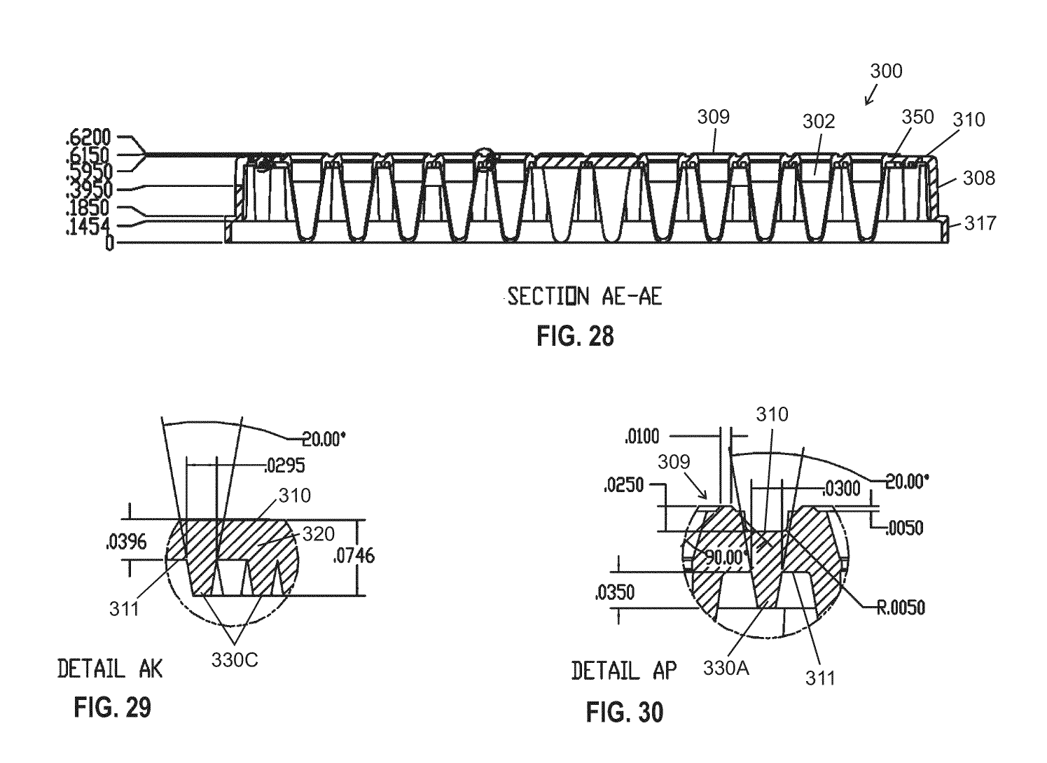

FIG. 28 is a cross-sectional view of the reinforced microplate 300 of FIG. 24 taken along section line AE-AE illustrated in FIG. 24. The array of wells 302 are defined in a deck that is peripherally bounded by a frame 308 that includes a peripheral skirt 317. Each well 302 includes a rim 309 that is raised relative to a top surface 310 of the deck, and sealing features 350 are provided between the array of wells 302 and (at least) corners of the microplate 300. FIG. 29 is a cross-sectional view of a peripheral deck portion of the reinforced microplate illustrated in FIG. 28, showing the square-shaped dots 330C protruding downward from a bottom surface 311 of the deck 320. FIG. 30 is a cross-sectional view of an upper central portion of the reinforced microplate 300 illustrated in FIG. 28 showing raised rim portions 309 of two adjacent microwells, and showing an inter-well rib 330A extending downward from the bottom surface 311 of the deck 320, which is further bounded by the top surface 310.

FIG. 31 is an end elevation view of a reinforced microplate 400 including elevated sealing features 450 and a peripheral frame 408 with an associated peripheral skirt 417 according to one embodiment, with omission of elevated well rim portions. The elevated sealing features 450 extend upward from a top surface 410 of a deck 420, and are preferably located proximate to corners of the microplate 400. Although two elevated sealing features 450 are shown in FIG. 31, in certain embodiments elevated sealing features may be provided in any suitable quantity of one, two, three, four, five, six, seven, eight, nine, ten or more. In certain embodiments, a single continuous or semi-continuous elevated sealing feature may be provided proximate to a perimeter of a deck of a microplate.

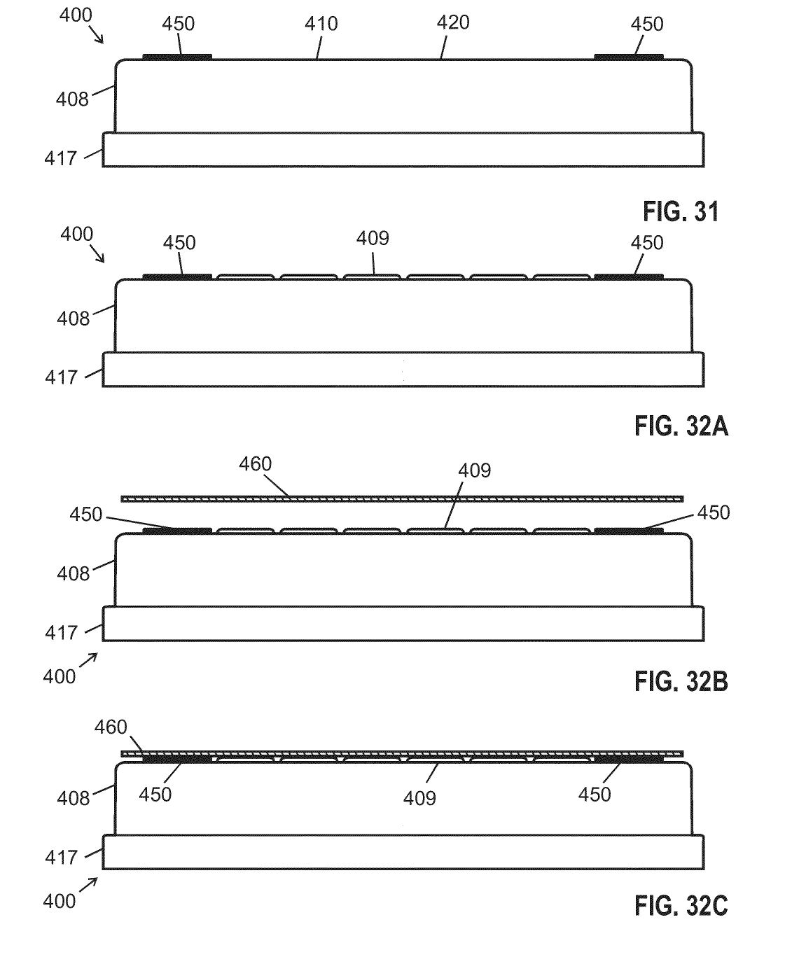

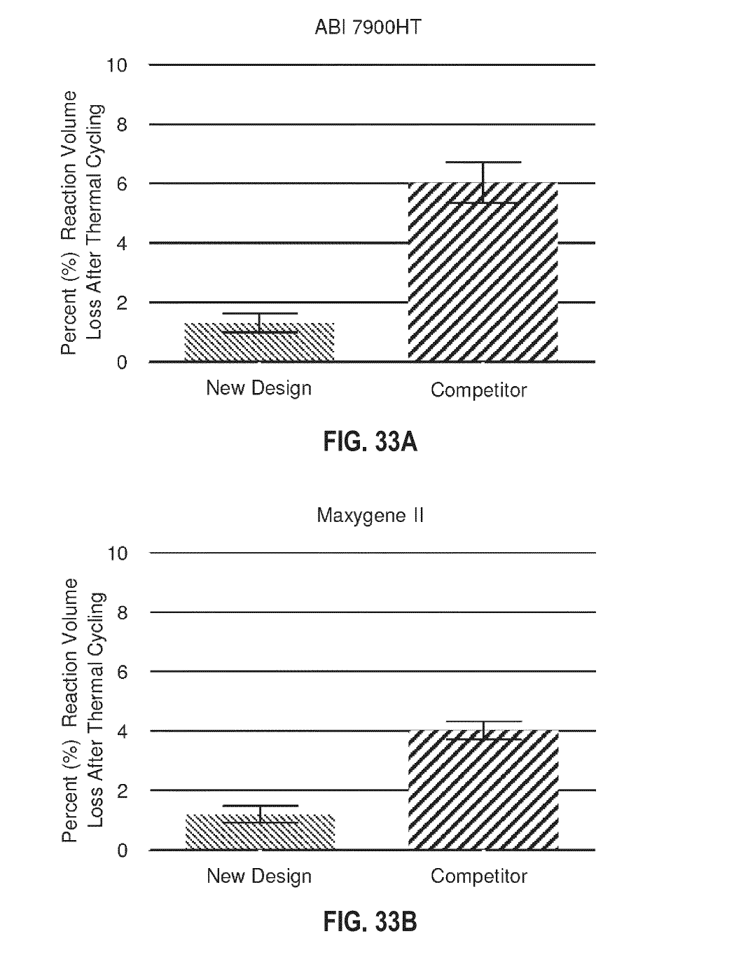

FIG. 32A is an end elevation view of a reinforced microplate 400 including elevated sealing features 450, raised rim portions 409 associated with microwells (not shown), and a peripheral frame 408 having an associated peripheral skirt 417 according to one embodiment. Preferably, the height of each raised rim portion 409 is substantially the same as the height of the elevated sealing features 450. FIG. 32B is an end elevation view of the reinforced microplate 400 of FIG. 32A arranged proximate to a sealing film 460 prior to application of the sealing film 460 to the reinforced microplate 400. FIG. 32C is an end elevation view of the reinforced microplate 400 of FIG. 33A arranged proximate to a sealing film 460 following application of the sealing film 460 to the reinforced microplate 400, wherein the sealing film 460 is arranged in contact with each raised rim portion 409 and the elevated sealing features 450.

FIGS. 33A-33B are bar graphs comparing sealing efficacy (in percent reaction volume loss after thermal cycling) for a film-covered half-skirt variety reinforced microplate with elevated sealing features and wells each having a raised rim as disclosed herein versus a conventional 4titude 4ti0900/C (4titude Limited, Wotton, Surrey, United Kingdom) film-covered microplate following thermal cycling in an ABI 7900 HT thermocycler and a Maxygene II thermocycler, respectively. Reaction volume loss was determined by weighing the microplates before and after thermal cycling. Three plates per thermal cycler were used in each instance, and the error bars in each figure represent standard error of the mean. In each instance, the reinforced microplate exhibited significantly less volume loss after thermal cycling.

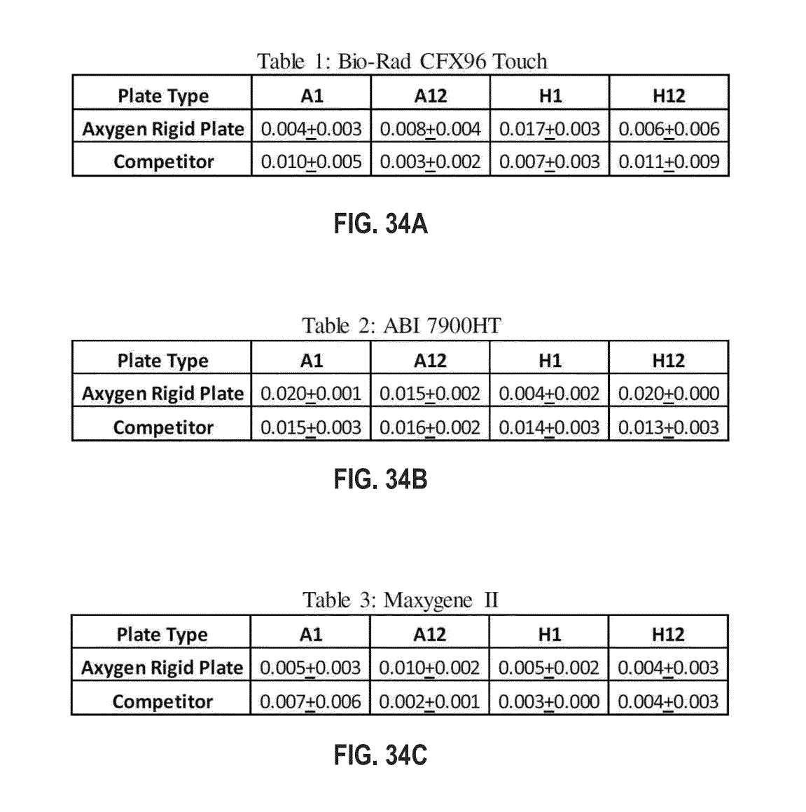

FIGS. 34A-34C are tables providing vertical displacement data (in inches) for microplate corners relative to a flat surface before and after thermal cycling for a reinforced microplate as disclosed herein and a conventional (4titude 4ti0900/C) microplate, for three different thermocyclers, respectively (namely, a Bio-Rad CFX96 Touch thermocycler in the case of FIG. 34A, an ABI 7900 HT thermocycler in the case of FIG. 34B, and a Maxygene II thermocycler in the case of FIG. 34C). Each microplate was evaluated for warping by measuring the distance between each corner of the microplate and a flat surface before and after thermal cycling. The four values A1, A12, H1, and H12 correspond to distances measured for the four corners of a microplate. All of the microplates displayed height change values of less than 0.02 inch, and did not exhibit visible warping regardless of which thermal cycler was used.

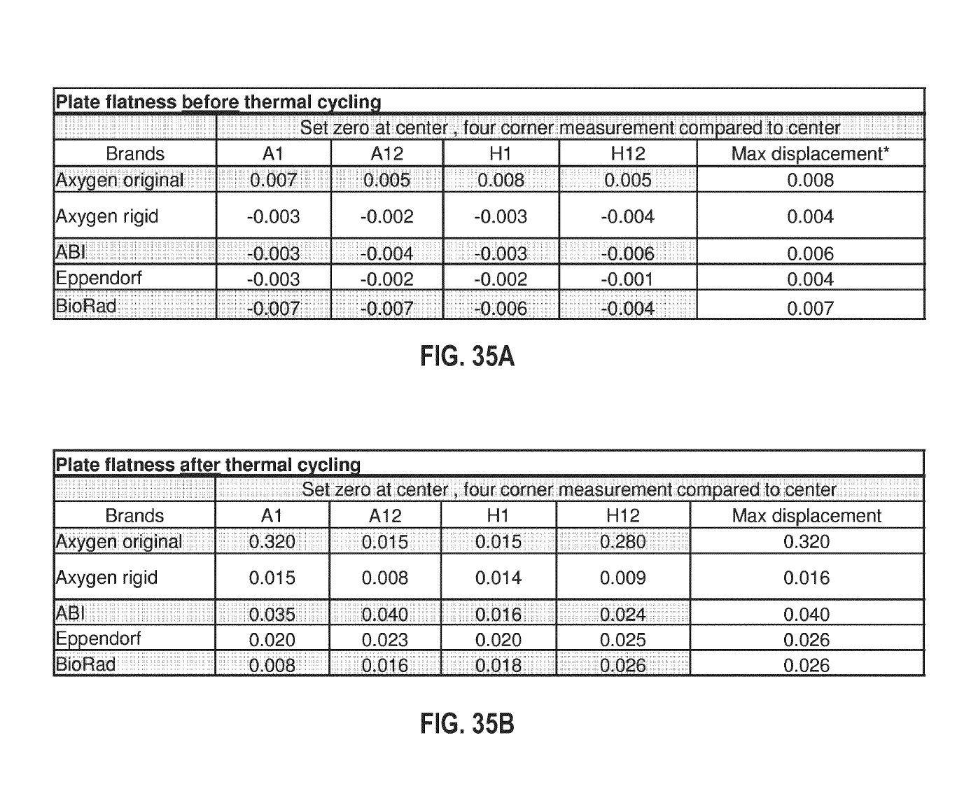

FIG. 35A is a table providing vertical distance relative to a center for four microplate corners (denoted as A1, A12, H1, H12) before thermocycling for a reinforced microplate (“Axygen rigid”, including with elevated sealing features and wells each having a raised rim) as disclosed herein and for four conventional microplates (Axygen original ABI, Eppendorf, and BioRad), respectively. A zero value was set at center, and the four corner measurement values A1, A12, H1, and H12 were compared to the center. The rightmost column of FIG. 35A provides a maximum displacement value of the four individual corner displacement values. FIG. 35B is a table providing vertical distance relative to a center for four microplate corners for the same microplates identified in FIG. 35A, but after thermocycling was complete. A zero value was set at center, and the four corner measurement values A1, A12, H1, and H12 were compared to the center. The rightmost column of FIG. 35B provides a maximum displacement value of the four individual corner displacement values. Notably, the rightmost values for the “Axygen original” and “Axygen rigid” microplates show that the microplate with enhanced reinforcement and sealing features (“Axygen rigid”) exhibits a 95% reduction in maximum corner displacement, with such maximum displacement also being lower than any of the remaining competitor microplates.

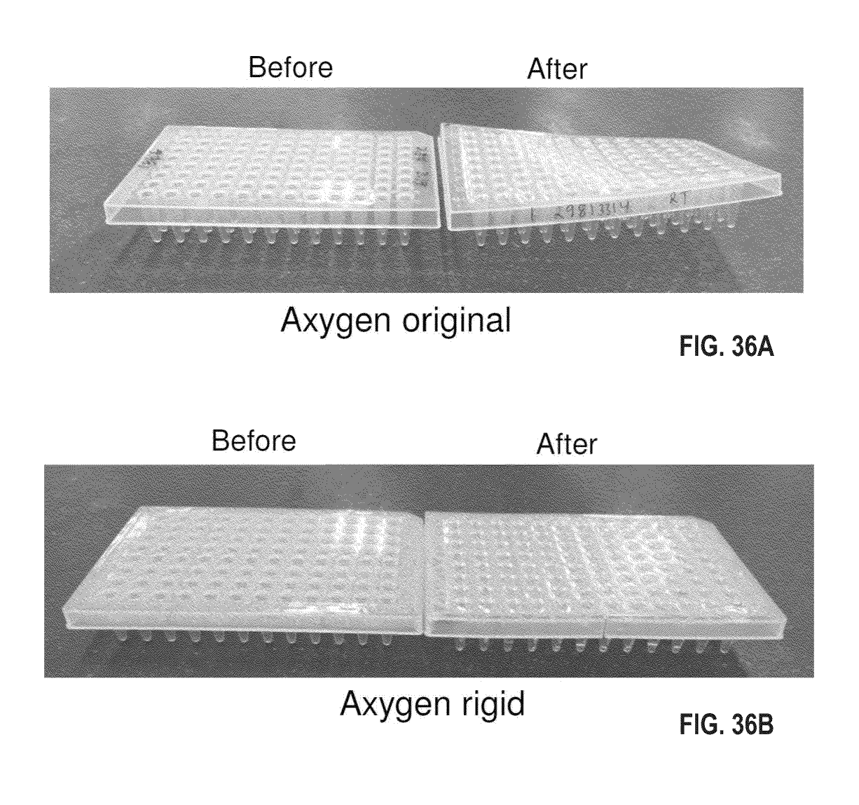

FIG. 36A is an upper perspective view photograph showing a first conventional “Axygen” microplate (at left) lacking reinforcement or sealing features disclosed herein before thermocycling, and showing a second conventional “Axygen” microplate (at right) after thermocycling. As shown, the conventional “Axygen” microplate exhibits significant warping after thermal cycling. For comparison, FIG. 36B is an upper perspective view photograph showing a first “Axygen rigid” reinforced microplate including reinforcement and sealing features as disclosed herein before thermocycling, and a second reinforced “Axygen rigid” microplate after thermocycling. As shown at right in FIG. 36B, the “Axygen rigid” microplate does not exhibit significant warping after thermal cycling, therefore representing a significant improvement over the second conventional “Axygen” microplate shown in FIG. 36A.

Various aspects and embodiments of the disclosure will be apparent following review of the preceding figures.

In accordance with one aspect of the disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. The peripheral deck portion comprises a plurality of locally increased peripheral deck thickness regions formed integral to the body, extending from the second surface, and configured to enhance stiffness of the microplate. Non-limiting examples of locally increased peripheral deck thickness regions include one or more reinforcing ribs (which may optionally be continuous or semi-continuous in nature) and/or a plurality of dots. In certain embodiments, a portion of each locally increased peripheral deck thickness region is arranged in contact with at least one other locally increased peripheral deck thickness region. In certain embodiments, the frame includes at least one wall comprising an outer surface and an inner surface, and the inner surface comprises a plurality of locally increased wall thickness regions (e.g., ribs extending in a generally vertical direction, optionally having a shape that tapers in a downward direction) configured to further enhance stiffness of the microplate. In certain embodiments, at least one stress-relieving slot defined in the frame in a direction substantially perpendicular to the deck, wherein the at least one slot may optionally be further defined in the peripheral deck portion. In certain embodiments, the deck, the plurality of wells, the frame, and the plurality of locally increased peripheral deck thickness regions are continuously formed of a same material composition. In certain embodiments, each well includes a raised rim with a top surface elevated above the first surface, an intermediate surface arranged between the top surface and the first surface, an inner transition surface extending between the intermediate surface and the top surface, and an outer transition surface extending between the intermediate surface and the top surface, wherein an angle between the inner transition surface and the top surface, as well as an angle between the outer transition surface and the top surface, is in a range of from 100 to 170 degrees. In certain embodiments, each well includes a raised rim with a top surface elevated above the first surface, and the microplate further comprises at least one elevated sealing feature extending from the first surface, and being arranged between corners of the deck and the plurality of wells.

In accordance another aspect of the present disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. The peripheral deck portion comprises a plurality of locally increased peripheral deck thickness regions formed integral to the body and comprising a plurality of dots, extending from the second surface, and configured to enhance stiffness of the microplate. In certain embodiments, at least a portion of each dot of a subset of the plurality of dots is arranged in contact with at least one other dot of the subset of dots, and/or comprises a substantially square cross-sectional shape taken along a plane parallel to the deck. In certain embodiments, at least one continuous or semi-continuous reinforcing rib extending from the second surface is further provided between the plurality of wells and the plurality of dots, and/or a plurality of ribs formed integral to the body and extending from the second surface is configured as a grid between different wells of the plurality of wells. In certain embodiments, the frame includes at least one wall comprising an outer surface and an inner surface, and the inner surface comprises a plurality of locally increased wall thickness regions (e.g., ribs extending in a generally vertical direction, optionally having a shape that tapers in a downward direction) configured to further enhance stiffness of the microplate. In certain embodiments, the deck, the plurality of wells, the frame, and the plurality of locally increased peripheral deck thickness regions are continuously formed of a same material composition. In certain embodiments, at least one stress-relieving slot defined in the frame in a direction substantially perpendicular to the deck, wherein the at least one slot may optionally be further defined in the peripheral deck portion. In certain embodiments, each well includes a raised rim with a top surface elevated above the first surface, an intermediate surface arranged between the top surface and the first surface, an inner transition surface extending between the intermediate surface and the top surface, and an outer transition surface extending between the intermediate surface and the top surface, wherein an angle between the inner transition surface and the top surface, as well as an angle between the outer transition surface and the top surface, is in a range of from 100 to 170 degrees. In certain embodiments, each well includes a raised rim with a top surface elevated above the first surface, and the microplate further comprises at least one elevated sealing feature extending from the first surface, and being arranged between corners of the deck and the plurality of wells.

In accordance with further aspects of the present disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. Additionally, the frame comprises at least one wall arranged non-parallel to the deck, the at least one wall comprises an outer surface and an inner surface, and the inner surface comprises a plurality of locally increased wall thickness regions configured to enhance stiffness of the microplate. In certain embodiments, each locally increased wall thickness region of the plurality of locally increased wall thickness regions includes a length extending in a generally vertical direction, and includes a width that tapers with increasing distance away from the second surface. In certain embodiments, the at least one wall is oriented in a generally vertical direction. In certain embodiments, the peripheral deck portion comprises a plurality of locally increased peripheral deck thickness regions formed integral to the body, extending from the second surface, and configured to enhance stiffness of the microplate. In certain embodiments, the plurality of locally increased peripheral deck thickness regions comprises a plurality of dots, optionally supplemented by at least one continuous or semi-continuous reinforcing rib arranged between the plurality of wells and the plurality of dots. In certain embodiments, the deck comprises a plurality of ribs formed integral to the body configured as a grid between different wells of the plurality of wells, extending from the second surface, and configured to enhance stiffness of the microplate. In certain embodiments, the deck, the plurality of wells, the frame, and the plurality of locally increased wall thickness regions are continuously formed of a same material composition. In certain embodiments, at least one stress-relieving slot defined in the frame in a direction substantially perpendicular to the deck, wherein the at least one slot may optionally be further defined in the peripheral deck portion. In certain embodiments, each well includes a raised rim with a top surface elevated above the first surface, an intermediate surface arranged between the top surface and the first surface, an inner transition surface extending between the intermediate surface and the top surface, and an outer transition surface extending between the intermediate surface and the top surface, wherein an angle between the inner transition surface and the top surface, as well as an angle between the outer transition surface and the top surface, is in a range of from 100 to 170 degrees. In certain embodiments, each well includes a raised rim with a top surface elevated above the first surface, and the microplate further comprises at least one elevated sealing feature extending from the first surface, and being arranged between corners of the deck and the plurality of wells.

In accordance with another aspect of the present disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. Each well of the plurality of wells comprises a raised rim including a top surface elevated above the first surface, an intermediate surface arranged between the top surface and the first surface, an inner transition surface extending between the intermediate surface and the top surface, and an outer transition surface extending between the intermediate surface and the top surface. Additionally, an angle between the inner transition surface and the top surface is in a range of from 100 to 170 degrees, and an angle between the outer transition surface and the top surface is in a range of from 100 to 170 degrees. In certain embodiments, the top surface, the intermediate surface, and the first surface are arranged along planes parallel to one another. In certain embodiments, the microplate further comprises at least one elevated sealing feature extending from the first surface, and being arranged between corners of the deck and the plurality of wells. In certain embodiments, the peripheral deck portion comprises a plurality of locally increased peripheral deck thickness regions formed integral to the body, extending from the second surface, and configured to enhance stiffness of the microplate. In certain embodiments, the plurality of locally increased peripheral deck thickness regions comprises a plurality of dots, optionally supplemented by at least one continuous or semi-continuous reinforcing rib arranged between the plurality of wells and the plurality of dots. In certain embodiments, the deck comprises a plurality of ribs formed integral to the body configured as a grid between different wells of the plurality of wells, extending from the second surface, and configured to enhance stiffness of the microplate. In certain embodiments, the frame comprises at least one wall arranged non-parallel to the deck, the at least one wall comprises an outer surface and an inner surface, and the inner surface comprises a plurality of locally increased wall thickness regions configured to enhance stiffness of the microplate. In certain embodiments, the deck, the plurality of wells, and the frame are continuously formed of a same material composition. In certain embodiments, at least one stress-relieving slot defined in the frame in a direction substantially perpendicular to the deck, wherein the at least one slot may optionally be further defined in the peripheral deck portion.

In accordance with additional aspects of the present disclosure, a microplate comprises a body including a first surface and an opposing second surface defining a deck, a plurality of wells extending from the second surface, a peripheral deck portion laterally surrounding the plurality of wells, and a frame surrounding the peripheral deck portion. Each well of the plurality of wells comprises a raised rim including a top surface elevated above the first surface, and the microplate further comprises at least one elevated sealing feature extending from the first surface, and being arranged between corners of the deck and the plurality of wells. In certain embodiments, the at least one elevated sealing feature comprises at least four elevated sealing features. In certain embodiments, the at least one elevated sealing feature comprises a same height as the top surface of the raised rim of each well of the plurality of wells. In certain embodiments, each well includes a raised rim with a top surface elevated above the first surface, an intermediate surface arranged between the top surface and the first surface, an inner transition surface extending between the intermediate surface and the top surface, and an outer transition surface extending between the intermediate surface and the top surface, wherein an angle between the inner transition surface and the top surface, as well as an angle between the outer transition surface and the top surface, is in a range of from 100 to 170 degrees. In certain embodiments, the peripheral deck portion comprises a plurality of locally increased peripheral deck thickness regions (e.g., a plurality of dots, optionally supplemented with at least one continuous or semi-continuous reinforcing rib arranged between the plurality of wells and the plurality of dots) formed integral to the body, extending from the second surface, and configured to enhance stiffness of the microplate. In certain embodiments, the deck comprises a plurality of ribs formed integral to the body configured as a grid between different wells of the plurality of wells, extending from the second surface, and configured to enhance stiffness of the microplate. In certain embodiments, the frame comprises at least one wall arranged non-parallel to the deck, the at least one wall comprises an outer surface and an inner surface, and the inner surface comprises a plurality of locally increased wall thickness regions configured to enhance stiffness of the microplate. In certain embodiments, the deck, the plurality of wells, the frame, and the at least one elevated sealing feature are continuously formed of a same material composition. In certain embodiments, at least one stress-relieving slot is defined in the frame and extends in a direction substantially perpendicular to the deck. In certain embodiments, at least one stress-relieving slot defined in the frame in a direction substantially perpendicular to the deck, wherein the at least one slot may optionally be further defined in the peripheral deck portion.

In further aspects of the disclosure, it is specifically contemplated that any two or more aspects, embodiments, or features disclosed herein may be combined for additional advantage.

As used herein, the singular forms “a,” “an” and “the” include plural referents unless the context clearly dictates otherwise. Thus, for example, reference to a “notch” includes examples having two or more such “notches” unless the context clearly indicates otherwise

The term “include” or “includes” means encompassing but not limited to, that is, inclusive and not exclusive.

“Optional” or “optionally” means that the subsequently described event, circumstance, or component, can or cannot occur, and that the description includes instances where the event, circumstance, or component, occurs and instances where it does not.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It is also noted that recitations herein refer to a component being “configured” or “adapted to” function in a particular way. In this respect, such a component is “configured” or “adapted to” embody a particular property, or function in a particular manner, where such recitations are structural recitations as opposed to recitations of intended use. More specifically, the references herein to the manner in which a component is “configured” or “adapted to” denotes an existing physical condition of the component and, as such, is to be taken as a definite recitation of the structural characteristics of the component.

While various features, elements or steps of particular embodiments may be disclosed using the transitional phrase “comprising,” it is to be understood that alternative embodiments, including those that may be described using the transitional phrases “consisting” or “consisting essentially of,” are implied. For example, implied alternative embodiments to a microplate comprising polypropylene include embodiments where a microplate consists of polypropylene and embodiments where a microplate consists essentially of polypropylene.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

1. A microplate comprising:
a body including a first surface and an opposing second surface defining a deck, a plurality of wells extending from the second surface, a peripheral deck portion laterally surrounding the plurality of wells, and a frame surrounding the peripheral deck portion;
wherein the peripheral deck portion comprises a plurality of locally increased peripheral deck thickness regions formed integral to the body, extending from the second surface, and configured to enhance stiffness of the microplate.

2. The microplate according to claim 1, wherein each locally increased peripheral deck thickness region of the plurality of locally increased peripheral deck thickness regions is arranged in contact with at least one other locally increased peripheral deck thickness region.

3. The microplate according to claim 1, wherein the deck comprises a plurality of ribs formed integral to the body configured as a grid between different wells of the plurality of wells, extending from the second surface, and configured to enhance stiffness of the microplate.

4. The microplate according to claim 1, wherein the plurality of locally increased peripheral deck thickness regions comprises a plurality of reinforcing ribs.

5. The microplate according to claim 1, wherein the plurality of locally increased peripheral deck thickness regions comprises a plurality of dots.

6. The microplate according to claim 5, wherein each dot of the plurality of dots comprises a substantially square cross-sectional shape taken along a plane parallel to the deck.

7. The microplate according to claim 5, wherein the plurality of locally increased peripheral deck thickness regions further comprises at least one continuous or semi-continuous reinforcing rib arranged between the plurality of wells and the plurality of dots.

8. The microplate according to claim 1, wherein the frame comprises at least one wall arranged non-parallel to the deck, the at least one wall comprises an outer surface and an inner surface, and the inner surface comprises a plurality of locally increased wall thickness regions configured to enhance stiffness of the microplate.

9. (canceled)

10. (canceled)

11. The microplate according to claim 1, wherein each well of the plurality of wells comprises a raised rim including a top surface elevated above the first surface, an intermediate surface arranged between the top surface and the first surface, an inner transition surface extending between the intermediate surface and the top surface, and an outer transition surface extending between the intermediate surface and the top surface; and
wherein an angle between the inner transition surface and the top surface is in a range of from 100 to 170 degrees, and an angle between the outer transition surface and the top surface is in a range of from 100 to 170 degrees.

12. The microplate according to claim 1, wherein each well of the plurality of wells comprises a raised rim including a top surface elevated above the first surface; and
wherein the microplate further comprises at least one elevated sealing feature extending from the first surface, and being arranged between corners of the deck and the plurality of wells.

13. The microplate according to claim 1, comprising at least one stress-relieving slot defined in the frame and extending in a direction substantially perpendicular to the deck.

14-26. (canceled)

27. A microplate comprising:
a body including a first surface and an opposing second surface defining a deck, a plurality of wells extending from the second surface, a peripheral deck portion laterally surrounding the plurality of wells, and a frame surrounding the peripheral deck portion;
wherein the frame comprises at least one wall arranged non-parallel to the deck, the at least one wall comprises an outer surface and an inner surface, and the inner surface comprises a plurality of locally increased wall thickness regions configured to enhance stiffness of the microplate.

28. The microplate according to claim 27, wherein each locally increased wall thickness region of the plurality of locally increased wall thickness regions includes a length extending in a generally vertical direction, and includes a width that tapers with increasing distance away from the second surface.

29. The microplate according to claim 27, wherein the at least one wall is oriented in a generally vertical direction.

30. The microplate according to claim 27, wherein the peripheral deck portion comprises a plurality of locally increased peripheral deck thickness regions formed integral to the body, extending from the second surface, and configured to enhance stiffness of the microplate.

31. The microplate according to claim 30, wherein the plurality of locally increased peripheral deck thickness regions comprises a plurality of dots.

32. The microplate according to claim 31, wherein the plurality of locally increased peripheral deck thickness regions further comprises at least one continuous or semi-continuous reinforcing rib arranged between the plurality of wells and the plurality of dots.

33. The microplate according to claim 27, wherein the deck comprises a plurality of ribs formed integral to the body configured as a grid between different wells of the plurality of wells, extending from the second surface, and configured to enhance stiffness of the microplate.

34. (canceled)

35. The microplate according to claim 27, wherein each well of the plurality of wells comprises a raised rim including a top surface elevated above the first surface, an intermediate surface arranged between the top surface and the first surface, an inner transition surface extending between the intermediate surface and the top surface, and an outer transition surface extending between the intermediate surface and the top surface; and
wherein an angle between the inner transition surface and the top surface is in a range of from 100 to 170 degrees, and an angle between the outer transition surface and the top surface is in a range of from 100 to 170 degrees.

36. The microplate according to claim 27, wherein each well of the plurality of wells comprises a raised rim including a top surface elevated above the first surface; and
wherein the microplate further comprises at least one elevated sealing feature extending from the first surface, and being arranged between corners of the deck and the plurality of wells.

37. The microplate according to claim 27, comprising at least one stress-relieving slot defined in the frame and extending in a direction substantially perpendicular to the deck.

38-49. (canceled)

50. A microplate comprising:
a body including a first surface and an opposing second surface defining a deck, a plurality of wells extending from the second surface, a peripheral deck portion laterally surrounding the plurality of wells, and a frame surrounding the peripheral deck portion;
wherein each well of the plurality of wells comprises a raised rim including a top surface elevated above the first surface; and
wherein the microplate further comprises at least one elevated sealing feature extending from the first surface, and being arranged between corners of the deck and the plurality of wells.

51. The microplate according to claim 50, wherein the at least one elevated sealing feature comprises at least four elevated sealing features.

52. (canceled)

53. The microplate according to claim 50, wherein each well of the plurality of wells comprises an intermediate surface arranged between the top surface and the first surface, an inner transition surface extending between the intermediate surface and the top surface, and an outer transition surface extending between the intermediate surface and the top surface; and
wherein an angle between the inner transition surface and the top surface is in a range of from 100 to 170 degrees, and an angle between the outer transition surface and the top surface is in a range of from 100 to 170 degrees.

54. The microplate according to claim 50, wherein the peripheral deck portion comprises a plurality of locally increased peripheral deck thickness regions formed integral to the body, extending from the second surface, and configured to enhance stiffness of the microplate.

55. The microplate according to claim 54, wherein the plurality of locally increased peripheral deck thickness regions comprises a plurality of dots.

56. The microplate according to claim 55, wherein the plurality of locally increased peripheral deck thickness regions further comprises at least one continuous or semi-continuous reinforcing rib arranged between the plurality of wells and the plurality of dots.

57. The microplate according to claim 50, wherein the deck comprises a plurality of ribs formed integral to the body configured as a grid between different wells of the plurality of wells, extending from the second surface, and configured to enhance stiffness of the microplate.

58. The microplate according to claim 50, wherein the frame comprises at least one wall arranged non-parallel to the deck, the at least one wall comprises an outer surface and an inner surface, and the inner surface comprises a plurality of locally increased wall thickness regions configured to enhance stiffness of the microplate.

59. (canceled)

60. The microplate according to claim 50, comprising at least one stress-relieving slot defined in the frame and extending in a direction substantially perpendicular to the deck.

61. (canceled)
